(19) United States
(12) Patent Application Publication
(10) Pub. No.: US 2009/0312688 A1
(43) Pub. Date: Dec. 17, 2009

(54) IONTOPHORESIS DRUG DELIVERY FORMULATION PROVIDING ACCEPTABLE SENSATION AND DERMAL ANESTHESIA

(75) Inventors: Vilambi NRK Reddy, Tamil/Nadu (IN); Preston Keusch, Jonesboro, ME (US)

Correspondence Address: K&L GATES LLP, 535 SMITHFIELD STREET, PITTSBURGH, PA, 15222, US

(73) Assignee: Vyteris, Inc., Fair Lawn, NJ (US)

(21) Appl. No.: 12/355,521

(22) Filed: Jan. 16, 2009

Related U.S. Application Data
(63) Continuation of application No. 11/537,141, filed on Sep. 29, 2006, now abandoned.
(60) Provisional application No. 60/722,641, filed on Sep. 30, 2005.

(51) Int. Cl. A61N 1/30; A61M 19/00

(52) U.S. Cl. 604/20

(57) ABSTRACT

A shelf-stable electrically assisted transdermal drug delivery system for highly effective electrotransport of an anesthetic and a vasoconstrictor producing clinically acceptable dermal anesthesia and sensation is provided. In certain embodiments the anesthetic includes lidocaine and the vasoconstrictor includes epinephrine. Medicament delivery is affected to provide dermal anesthesia with little or no sensation during delivery, as measured by a variety of indicator tests. Methods of producing dermal anesthesia in patients are also provided.

### CROSS REFERENCE TO RELATED APPLICATIONS

This application claims priority under 35 U.S.C. § 119(e) to U.S. Provisional Application Ser. No. 60/722,641, filed Sep. 30, 2005, the disclosure of which is incorporated herein by reference in its entirety.

### BACKGROUND

1. Field of the Invention

Highly shelf-stable electrically assisted transdermal drug delivery systems for producing acceptable delivery, sensation, and dermal anesthesia, and uses therefor.

2. Description of the Related Art

Transdermal drug delivery systems have become an increasingly important means of administering drugs. Such systems offer advantages clearly not achievable by other modes of administration such as avoiding introduction of the drug through the gastro-intestinal tract or punctures in the skin, to name a few.

There are two types of transdermal drug delivery systems: “passive” and “active.” Passive systems deliver drug through the skin of the user unaided, an example of which would involve the application of a topical anesthetic to provide localized relief, as disclosed in U.S. Pat. No. 3,814,095. Active systems, on the other hand, use external force to facilitate delivery of a drug through a patient's skin. Examples of active systems include ultrasound, electroosmosis and/or iontophoresis.

Iontophoretic delivery of a medicament is accomplished by application of a voltage to a medicament-loaded reservoir-electrode, sufficient to maintain a current between the medicament-loaded reservoir-electrode and a return electrode (another electrode) applied to a patient's skin so that an ionic form of the desired medicament is delivered to the patient.

Conventional iontophoretic devices, such as those described in U.S. Pat. Nos. 4,820,263; 4,927,408; and 5,084,008, the disclosures of which are hereby incorporated by reference, deliver a drug transdermally by iontophoresis. These devices typically contain two electrodes—an anode and a cathode. In a typical iontophoretic device, electric current is driven from an external power supply. In a device for delivering drug from an anode, positively charged drug is delivered into the skin at the anode, with the cathode completing the electrical circuit. Likewise, in a system for delivering drug from a cathode, negatively charged drug is delivered into the skin at the cathode, with the anode completing the electrical circuit. Accordingly, there has been considerable interest in iontophoresis to perform delivery of drugs for a variety of purposes. One example is the delivery of lidocaine, a common topical, local anesthetic.

A number of iontophoretic systems have been described for delivery of lidocaine in order to affect dermal anesthesia. Concomitant delivery of both lidocaine and a vasoconstrictor, such as epinephrine has been found to be most desirable. The vasoconstrictor retards the migration of the lidocaine from the site of delivery.

International Patent Publication WO 98/208869 discloses an iontophoretic device for delivery of epinephrine and lidocaine HCl. Likewise, U.S. Pat. Nos. 4,786,277 and 6,295,469; and EP 0941 085 B1 disclose iontophoresis devices for delivery of lidocaine. Commercial products exist for delivery of lidocaine and epinephrine. For example, Iomed, Inc. markets iontophoretic systems including an iontophoresis system called Numby Stuff® (a registered trademark of Iomed, Inc. of Salt Lake City, Utah) for local delivery of lidocaine and epinephrine by iontophoresis. That device comprises the Phoresor® Iontophoretic Drug Delivery System controller and is marketed as a kit containing active and return electrode pairs (Phoresor® is a registered trademark of Iomed, Inc. of Salt Lake City, Utah). A multiple-use vial of a solution of 2% lidocaine HCl and 1:100,000 epinephrine (under the trade name Iontocaine®, a registered trademark of Iomed, Inc. of Salt Lake City, Utah) is included with the kit. The system has to be assembled and the liquid containing lidocaine and epinephrine is then added to the active patch just before use. It is easy for a practitioner to lose track of the age of the multi-use vial of lidocaine and epinephrine, consequently allowing the epinephrine to degrade in the vial. It also is cumbersome to preload a patch just before use. A syringe is needed for each use and the potential for dose-to-dose variation is present. For example, the loading syringe may not be filled with the proper amount of solution, some of the solution may not be applied to the patch and/or the liquid can squeeze out of the absorbent drug containing electrode because the solution is a separate phase from the absorbent reservoir.

One technical hurdle in the iontophoretic delivery of lidocaine and epinephrine is achieving the combination of rapid and effective dermal anesthesia without causing patient discomfort. Lidocaine and epinephrine formulations are known in the art that deliver up to 2% lidocaine with very low amounts of epinephrine in the ratio of 1:100,000. To achieve dermal anesthesia, the applied current in such a device either needs to be uncomfortably high, or the lower current must be applied for longer periods of time. High charge densities (traditionally in the range of greater than 2.0 milliAmp·minutes/centimeters² (mA·min/cm²) and high drug concentrations are known to cause discomfort during delivery and often result in such combined side effects as erythema and edema after drug delivery. To date, there are no teachings on how to make a high-current, packaged, preloaded, high concentration iontophoretic device for comfortably delivering lidocaine and epinephrine in a minimal time, while producing acceptable dermal anesthesia.

### SUMMARY OF INVENTION

Provided is a shelf-storage stable iontophoretic device for delivery of a topical anesthetic, such as lidocaine in combination with a vasoconstrictor, such as epinephrine, providing acceptable dermal anesthesia and sensation. In the device, the drug is stored as a solid solution in a solid solution reservoir thereby avoiding squeezing out of drug and changes in the active area of the reservoir. The device includes an electrode and a hydrophilic polymeric reservoir situated in electrically conductive relation to the electrode and is ready for use immediately upon removal from its packaging—there is no need to load the active ingredients in the anode or return solution in the cathode. The device is pharmaceutically, chemically, electrochemically and physically stable for more than 24 months at room-temperature, with stability for extended periods at elevated temperatures, making manufacture, distribution and storage more effective and providing the end user a greater confidence in the product, with less returns of the device from customer. Further, the device provides acceptable levels of dermal anesthesia after a short delivery time of less than about ten minutes at high current fluxes, with minimal unpleasant or painful sensation during delivery.

According to one non-limiting embodiment, the present disclosure provides for an iontophoresis electrode assembly comprising an anode assembly comprising a pre-loaded hydrogel drug reservoir in electrical communication with a first electrode. The reservoir comprises (a) a vasoconstrictor; and (b) an anesthetic. The iontophoresis electrode assembly produces clinically acceptable dermal anesthesia and sensation at a treated site as measured by at least about a 50% reduction of dermal sensitivity to an applied force and produces at least one of: (a) a von Frey score of at least a DELTAI0 of 1.15; (b) a hedonic score of greater than about −1.5 on a visual analog scale (VAS) ranging from −10 to 10; (c) a pain cannulation score of less than about 51 mm on a visual analog scale (VAS) ranging from 0 to 100 mm; (d) a Sensation Intensity Scale (SIS) score of less than about 7.4 on a scale of 0 to 4, and resolving in less than about 48 hours; (e) a Draize score for erythema of less than about 2 on a scale from 0 to 4, and resolving in less than about 48 hours; and (f) a Draize score for edema of less than about 1 on a scale from 0 to 4, and resolving in less than about 48 hours. The above aspects are produced with an applied charge density of between about 1.5 mA·min/cm² and about 4.2 mA·min/cm² that is applied for less than about 10 minutes.

Another non-limiting embodiment of the present disclosure provides for a method of producing local anesthesia in a patient. The method comprises applying a charge density of at least about 1.5 mA·min/cm² for at least about 5 minutes to an electrically assisted drug delivery system comprising an anode assembly. The anode assembly includes a pre-loaded hydrogel drug reservoir in electrical contact with the patient, and the drug reservoir comprises an anesthetic and a vasoconstrictor. The electrically assisted drug delivery system produces clinically acceptable dermal anesthesia and sensation at a treated site as measured by at least about a 50% reduction of dermal sensitivity to an applied force and produces at least one of: (a) a von Frey score of at least a DELTAI0 of 1.15; (b) a hedonic score of greater than about −1.5 on a VAS ranging from −10 to 10; (c) a pain cannulation score of less than about 51 mm on a VAS ranging from 0 to 100 mm; (d) a Sensation Intensity Scale score of less than about 7.4 on a scale of 0 to 4, and resolving in less than about 48 hours; (e) a Draize score for erythema of less than about 2 on a scale from 0 to 4, and resolving in less than about 48 hours and (f) a Draize score for edema of less than about 1 on a scale from 0 to 4, and resolving in less than about 48 hours.

### BRIEF DESCRIPTION OF THE DRAWINGS

FIG. 1 (prior art) shows schematically an electrically assisted drug delivery system including an anode assembly, a cathode assembly and a controller/power supply.

FIG. 2 shows an exploded isometric view of various aspects of an integrated electrode assembly provided in accordance with the present invention.

FIG. 3 shows an exploded isometric view of various aspects of an integrated electrode assembly provided in accordance with the present invention.

FIG. 4 shows an elevated view of various aspects of an integrated electrode assembly provided in accordance with the present invention.

FIG. 5A includes an exploded isometric view illustrating various aspects of the interconnection of an integrated electrode assembly provided in accordance with the present invention with components of an electrically assisted delivery device.

FIG. 5B shows a schematic representation of the interaction between a portion of an integrated electrode assembly provided in accordance with the present invention and components of an electrically assisted delivery device.

FIG. 5C illustrates a schematic representation of the interaction between a portion of an integrated electrode assembly provided in accordance with the present invention and components of an electrically assisted delivery device.

FIG. 6 includes a schematic elevated view of various aspects of an integrated electrode assembly provided in accordance with the present invention.

FIGS. 6B and 6C show cross-sectional views illustrating aspects of the electrode assembly of FIG. 6.

FIG. 7 includes a schematic elevated view of various aspects of the release cover of an integrated electrode assembly provided in accordance with the present invention.

FIG. 7A includes a cross-sectional view of the release cover of FIG. 7.

FIG. 8 includes a schematic that illustrates the effect of electrode geometry and spacing on the delivery paths of a composition through a membrane.

FIG. 9 includes a schematic that illustrates the effect of electrode geometry and spacing on the delivery paths of a composition through a membrane.

FIG. 10 shows a cross-sectional view of a schematic unloaded electrode assembly in contact with a loading solution.

FIG. 11 is a cut-away view of a package including an electrode assembly structured in accordance with the present invention.

### DETAILED DESCRIPTION

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

Unless otherwise specified, embodiments of the present invention are employed under “normal use” conditions, which refer to use within standard operating parameters for those embodiments. During operation of various embodiments described herein, a deviation from a target value of one or more parameters of about +110% or less for an iontophoretic device under “normal use” is considered an adequate deviation for purposes of the present invention.

Described herein is an electrode assembly for electrically assisted transmembrane delivery of drugs, for example lidocaine and epinephrine, with acceptable dermal anesthesia and sensation. The electrode assembly exhibits exceptional shelf-stability, even at temperatures greater than room temperature (25° C.).

The terms “unloaded” or “unloaded reservoir,” are necessarily defined by the process of loading a reservoir. In the loading process, a drug or other compound or composition if absorbed, adsorbed and/or diffused into a reservoir to reach a final content or concentration of the compound or composition. An unloaded reservoir is a reservoir that lacks that compound or composition in its final content or concentration. In one example, the unloaded drug reservoir is a hydrogel, as described in further detail below, which includes water and a salt. One or more additional ingredients may be included in the unloaded reservoir. Typically, active ingredients are not present in the unloaded gel reservoir. Other additional, typically non-ionic ingredients, such as preservatives, may be included in the unloaded reservoir. Although the salt may be one of many salts, including alkaline metal halide salts, the salt typically is sodium chloride. Other halide salts such as, without limitation, KCl or LiCl might be equal to NaCl in terms of functionality, but may not be preferred. Use of halide salts to prevent electrode corrosion is disclosed in U.S. Pat. Nos. 6,629,968 and 6,635,045 both of which are incorporated herein by reference in their entireties.

The term “electrically assisted delivery” refers to the facilitation of the transfer of any compound across a membrane, such as, without limitation, skin, mucous membranes and nails, by the application of an electric potential across that membrane. “Electrically assisted delivery” is intended to include, without limitation, iontophoretic, electrophoretic and electroendosmotic delivery methods. By “active ingredient,” it is meant, without limitation, drugs, active agents, therapeutic compounds, medicament, and any other compound capable of eliciting any pharmacological effect in the recipient that is capable of transfer by electrically assisted delivery methods. A “transdermal device” or “transdermal patch” includes both active and passive transdermal devices or patches.

The term “lidocaine,” unless otherwise specified, refers to any water-soluble form of lidocaine, including salts or derivatives, homologs or analogs thereof, so long as they can be solubilized in an aqueous solution and be present in a substantially ionic form. For example, as is used in the Examples below, “lidocaine” refers to lidocaine hydrochloride (HCl), in substantially ionic form, commercially available as XYLOCAINE® (a trademark of AstraZeneca LP of Wayne, Pa.), among other names.

The term “epinephrine” refers to any form of epinephrine, salts, its free base or derivatives, homologs or analogs thereof so long as they can be solubilized in an aqueous solution and be present in a substantially ionic form. For example, as is used in the examples below, “epinephrine” refers to epinephrine bitartrate, in substantially ionic form.

As applied to various embodiments of electrically assisted delivery devices described herein, the term “integrated” as used in connection with a device indicates that at least two electrodes are associated with a common structural element of the device. For example, and without limitation, a transdermal patch of an iontophoretic device may include both a cathode and an anode “integrated” therein, i.e., the cathode and anode are attached to a common backing.

As applied to various embodiments of electrically assisted delivery devices described herein, a “flexible” material or structural component is generally compliant and conformable to a variety of membrane surface area configurations and a “stiff” material or structural component is generally not compliant and not conformable to a variety of membrane surface area configurations. In addition, a “flexible” material or component possesses a lower flexural rigidity in comparison to a “stiff” material or structural component having a higher flexural rigidity. For example and without limitation, a flexible material when used as a backing for an integrated patch can substantially conform over the shape of a patient's forearm or inside elbow, whereas a comparatively “stiff” material would not substantially conform in the same use as a backing.

As applied herein, the term “transfer absorbent” includes any media structured to retain therein a fluid or fluids on an at least temporary basis and to release the retained fluids to another medium such as a hydrogel reservoir, for example. Examples of “transfer absorbents” that may be employed herein include, without limitation, non-woven fabrics and open-cell sponges and foams.

As used herein, “stable” and “stability” refer to a property of individual packaged electrode-reservoir assemblies, and typically is demonstrated statistically. The term “stable” refers to retention of a desired quality, with particular, but not exclusive focus on active ingredients such as epinephrine content, lidocaine content, within a desired range. For example, in an iontophoretic device, the U.S. Food and Drug Administration (FDA) may require retention, as a lot, of 90% of the label claim of epinephrine over a given time period using a least square linear regression statistical method with a 95% confidence level. As used herein, however, an electrode assembly and/or parts thereof, are considered stable so long as they substantially retain their desired function in an iontophoretic system. Stability, though measured by any applicable statistical method, is a quality of the electrode assembly. Therefore, methods other than FDA-approved statistical methods may be used to quantitatively assess stability. For instance, even though for FDA purposes, a 95% confidence level may be required, those limits are not literally required for a device to be called “stable.” Similarly, and for exemplary purposes only, a “stable” iontophoretic electrode may be said to retain 80% of the original epinephrine concentration over a given time period, as determined by least square linear regression analysis.

As used generally herein, an electrode-reservoir, reservoir or electrode assembly is stable when hermetically sealed for a given time period. This means that when the electrode assembly is sealed in a container that is impermeable to oxygen and water (“hermetically sealed”), the electrode-reservoir retains a specified characteristic or parameter within desired boundaries for a given time period. By “original concentration”, “original amounts” or “original levels” it is meant the concentration, amount or level of any constituent or physical, electrochemical or electrical parameter relating to the electrode assembly at a time point designated as t=0, and typically refers to a time point after the electrode assembly is sealed within the hermetically sealed container. This time may take up to a few weeks to ensure uniform distribution of ingredients in the reservoir(s).

As used herein, “anesthesia” refers to a state characterized by a loss of sensation as a result of pharmacologic depression of nerve function.

As used herein, “non-necrotizing” refers to not causing necrosis, wherein necrosis is defined as death of tissues or cells caused when not enough blood is supplied to the tissues or cells. With particular reference to “non-necrotizing amount of vasoconstrictor,” the amount of vasoconstrictor delivered in the invention does not cause the tissue in contact with the vasoconstrictor to be injured to the point wherein blood supply is substantially compromised causing cellular death.

### Iontophoretic Device

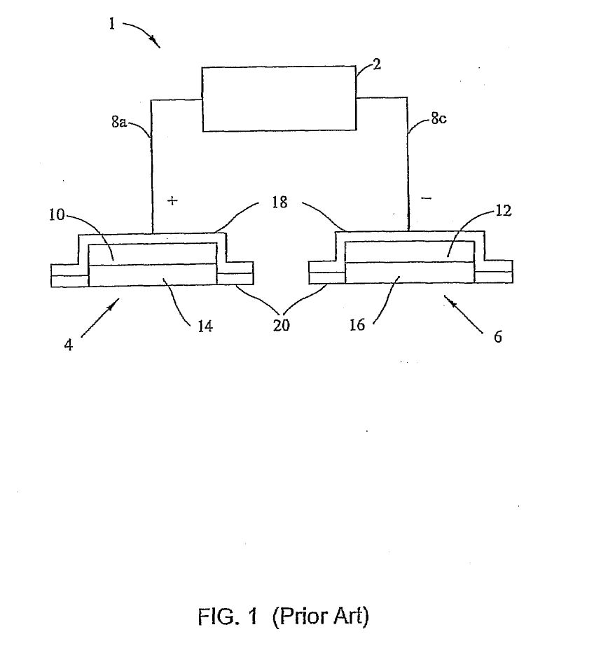

FIG. 1 depicts schematically a typical electrically assisted drug delivery apparatus 1. The apparatus 1 includes an electrical power supply/controller 2, an anode electrode assembly 4 and a cathode electrode assembly 6. Anode electrode assembly 4 and cathode electrode assembly 6 are connected electrically to the power supply/controller 2 by conductive leads 8a and 8c (respectively). The anode electrode assembly 4 includes an anode 10 and the cathode electrode assembly 6 includes a cathode 12. The anode 10 and the cathode 12 are both in electrical contact with the leads 8a, 8c. The anode electrode assembly 4 further includes an anode reservoir 14, while the cathode electrode assembly 6 further includes a cathode reservoir 16. Both the anode electrode assembly 4 and the cathode electrode assembly 6 include a backing 18 to which a pressure sensitive adhesive 20 is applied in order to affix the electrode assemblies 4, 6 to a membrane (e.g., skin of a patient), to establish electrical contact for the reservoirs 14, 16 with the membrane. Optionally, the reservoirs 14, 16 may be at least partially covered with the pressure sensitive adhesive 20.

### Platform I:

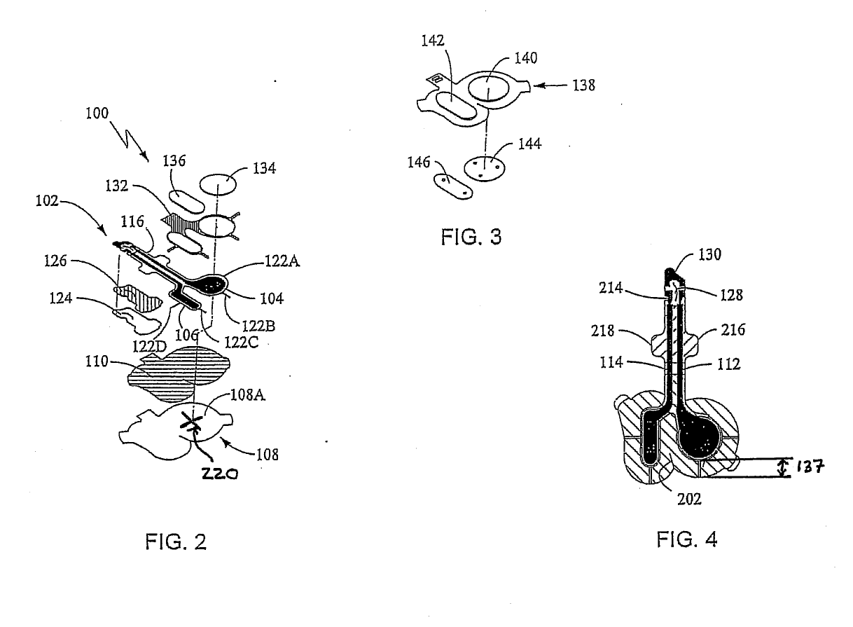

FIGS. 2 through 10 illustrate various aspects of an integrated electrode assembly 100 of the present invention structured for use with an electrically assisted delivery device, for example, for delivery of a composition through a membrane. A printed electrode layer 102 including two electrodes (an anode 104 and a cathode 106) is connected to a flexible backing 108 by a layer of flexible transfer adhesive 110 positioned between the printed electrode layer 102 and the flexible backing 108. One or more leads 112, 114 may extend from the anode 104 and/or cathode 106 to a tab end portion 116 of the printed electrode layer 102. In various aspects, an insulating dielectric coating 118 may be deposited on and/or adjacent to at least a portion of one or more of the electrodes 104, 106 and/or the leads 112, 114. The dielectric coating 118 may serve to strengthen or bolster the physical integrity of the printed electrode layer 102; to reduce point source concentrations of current passing through the leads 112, 114 and/or the electrodes 104, 106; and/or to resist creating an undesired short circuit path between portions of the anode 104 and its associated lead 112 and portions of the cathode 106 and its associated lead 114.

In other aspects, one or more splines 122 (122A, 122B, 122C, 122D) may be formed to extend from various portions of the printed electrode layer 102, as shown. It can be seen that at least one advantage of the splines 122 is to facilitate manufacturability (e.g., die-cutting of the electrode layer 102) and construction of the printed electrode layer 102 for use in the assembly 100. The splines 122 may also help to resist undesired vacuum formation when a release cover (see discussion hereafter) is positioned in connection with construction or use of the assembly 100.

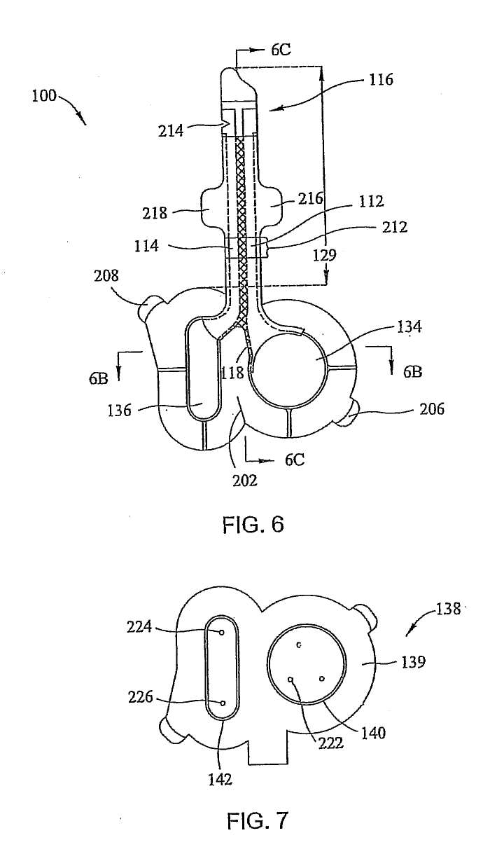

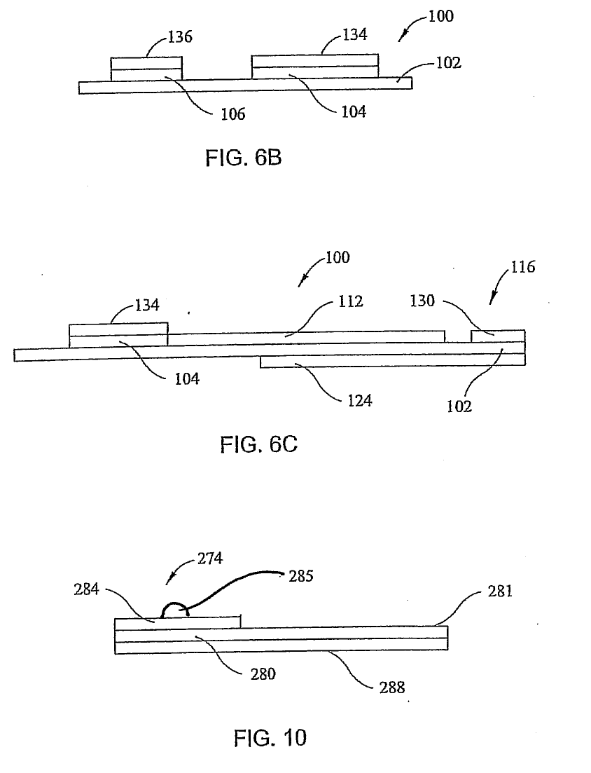

In other embodiments of the present invention, a tab stiffener 124 is connected to the tab end portion 116 of the printed electrode layer 102 by a layer of adhesive 126 positioned between the tab stiffener 124 and the tab end portion 116. In various embodiments, a tab slit 128 may be formed in the tab end portion 116 of the assembly 100 (as shown more particularly in FIGS. 2 and 4). The tab slit 128 may be formed to extend through the tab stiffener 124 and the layer of adhesive 126. In other embodiments, a minimum tab length 129 (as shown particularly in FIG. 6) as structured in association with the tab end portion 116 may be in the range of at least about 3.81 cm (1.5 inches).

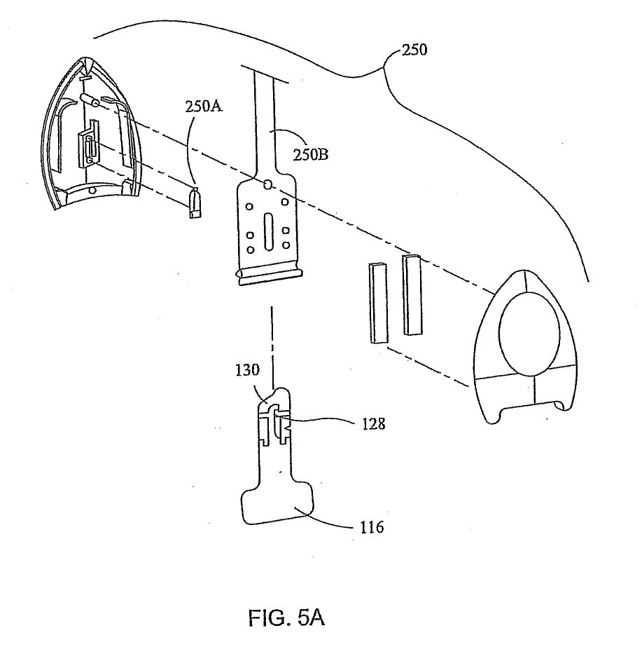

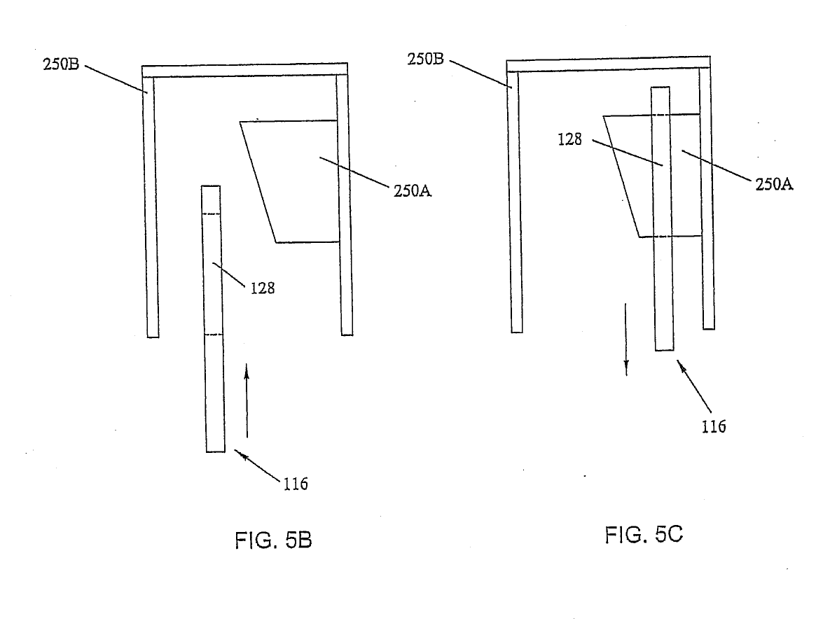

With reference to FIGS. 5A-5C, the tab end portion 116 may be structured to be mechanically or electrically operatively associated with one or more components of an electrically assisted drug delivery device such as a knife edge 250A of a connector assembly 250, for example. As shown schematically in FIGS. 5B and 5C, once the tab end portion 116 is inserted into a flexible circuit connector 250B of the connector assembly 250, the tab slit 128 of the tab end portion 116 may be structured to receive therein the knife edge 250A. It can be appreciated that the interaction between the knife edge 250A and the tab slit 128 may serve as a tactile sensation aid for a user manually inserting the tab end portion 116 into the flexible circuit connector 250B of the connector assembly 250. In addition, the knife edge 250A may be structured, upon removal of the tab end portion 116 from the connector assembly 250, to cut or otherwise disable one or more electrical contact portions positioned on the tab end portion 116, such as a sensor trace 130, for example. It can be seen that this disablement of the electrical contact portions may reduce the likelihood that unintended future uses of the assembly 100 will occur after an initial use of the assembly 100 and the connector assembly 250 for delivery of a composition through a membrane, for example.

In other aspects, a layer of transfer adhesive 110 may be positioned in communication with the printed electrode layer 102 to facilitate adherence and/or removal of the assembly 100 from a membrane, for example, during operation of an electrically assisted delivery device that includes the assembly 100. Optionally a second adhesive layer 132 may be positioned on the electrode layer to peripherally surround the printed electrode layer 102 and to further facilitate adherence and/or removal of the assembly 100 from a membrane. As shown in FIG. 2, a first hydrogel reservoir 134 is positioned for electrical communication with the anode 104 of the printed electrode layer 102 and a second hydrogel reservoir 136 is positioned for electrical communication with the cathode 106 of the printed electrode layer 102. In other aspects, although a hydrogel may be preferred in many instances, there may be substantially no hydrogel reservoir associated with the cathode 106, or a substance including NaCl, for example, may be associated with the cathode 106.

As shown in FIG. 3, a release cover 138 includes an anode-donor absorbent well 140 and a cathode-return absorbent well 142. The anode-donor portion 140 is structured to receive therein a donor transfer absorbent 144 suitably configured/sized for placement within the anode-donor portion 140. Likewise, the cathode-return portion 142 is structured to receive therein a return transfer absorbent 146 suitably configured/sized for placement within the cathode-return portion 142. The transfer absorbents 144, 146 may be attached to their respective portions 140, 142 by a suitable method or apparatus, such as by use of one or more spot welds, for example. In construction of the assembly 100, it can be seen that the release cover 138 is structured for surface contact with the flexible transfer adhesive layer 110 such that the donor transfer absorbent 144 establishes contact with the hydrogel reservoir 134 associated with the anode 104 and the return transfer absorbent 146 establishes contact with the hydrogel reservoir 136 associated with the cathode 106.

In various embodiments, the integrated assembly 100 may include a first reservoir-electrode assembly (including the reservoir 134 and the anode 104) charged with lidocaine HCl and epinephrine bitartrate, for example, that may function as a donor assembly and a second reservoir-electrode assembly (including the reservoir 136 and the cathode 106) that may function as a return assembly. The assembly 100 includes the reservoir-electrode 104 and the reservoir-electrode 106 mounted on an electrode assembly securement portion 108A of the flexible backing 108. The assembly 100 includes two electrodes, an anode 104 and a cathode 106, each having an electrode surface and an operatively associated electrode trace or lead 112 and 114, respectively. The electrodes 104, 106 and the electrode traces 112, 114 may be formed as a thin film deposited onto the electrode layer 102 by use of a conductive ink, for example. The conductive ink may include Ag and Ag/AgCl, for example, in a suitable binder material, and the conductive ink may have the same composition for both the electrodes 104, 106 and the electrode traces 112, 114. A substrate thickness for the conductive ink may be in the range of about 0.005 cm (0.002 inches) to 0.018 cm (0.007 inches). In other aspects, the specific capacity of the conductive ink is preferably in the range of about 2 to 120 mA·min/cm², or more preferably in the range of 5 to 20 mA·min/cm². In various aspects, the conductive ink may comprise a printed conductive ink. The electrodes 104, 106 and the electrode traces 112, 114 may be formed in the electrode layer 102 to comprise a stiff portion of the assembly 100.

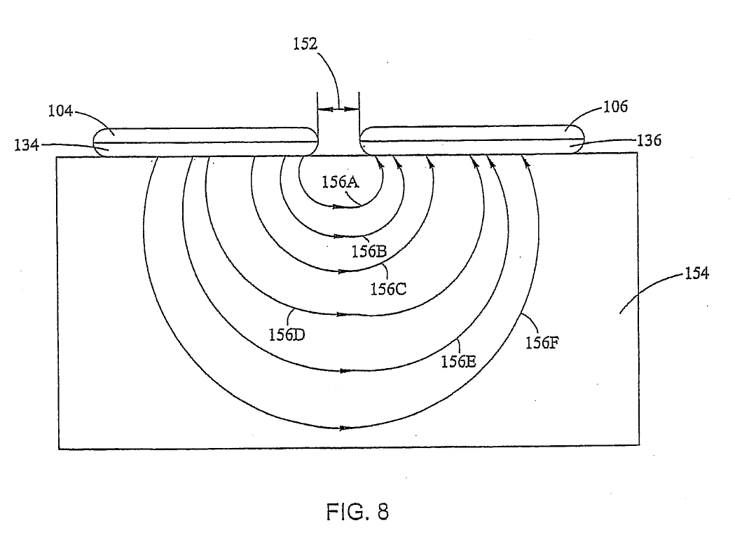

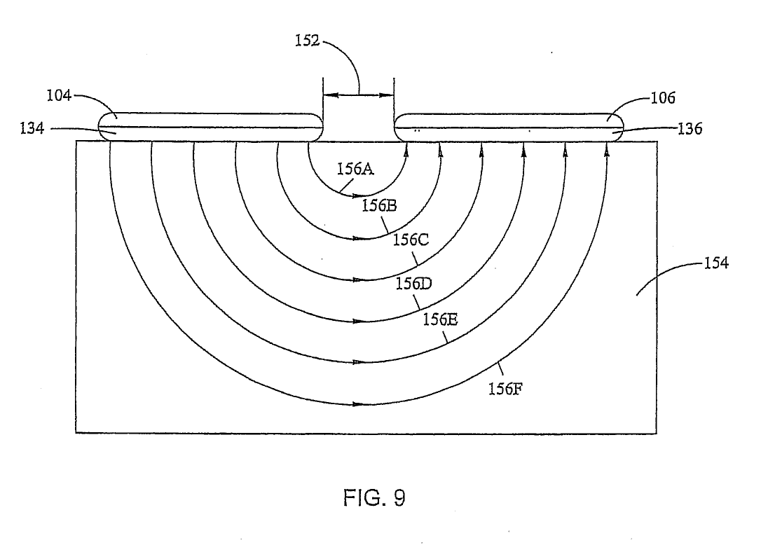

In various embodiments of the present invention, a shortest distance 152 between a surface area of the anode 104/reservoir 134 assembly and a surface area of the cathode 106/reservoir 136 assembly may be in the range of at least about 0.65 cm (0.25 inches). Referring now to FIG. 8, for example, it can be seen that inappropriate selection of the distance 152, the geometric configuration of the electrodes 104, 106 (e.g., thickness, width, total surface area, and others), and/or a combination of other factors may result in a substantially non-uniform delivery of a composition between the electrodes through a membrane 154 during operation of the assembly 100. As shown, the delivery of the composition through the membrane is shown schematically by composition delivery paths 156A-156F. In contrast, as shown in FIG. 9, appropriate selection of the distance 152, the geometric configuration of the electrodes 104, 106 (e.g., thickness, width, total surface area, and others), and/or a combination of other factors may result in a substantially uniform delivery of a composition between the electrodes through a membrane 154 as shown by delivery paths 156A-156F. It can be seen that the inventors have recognized the problem of delivering a composition through a membrane that may include scar tissue, for example, or another variation in the density of the membrane that may adversely impact the effectiveness and uniformity of delivery of the composition between the electrodes of a device, for example.

In accordance with the discussion above, the electrodes 104, 106 may each be mounted with bibulous reservoirs 134, 136 (respectively) formed from a cross-linked polymeric material such as cross-linked poly(vinylpyrrolidone) hydrogel, for example, including a substantially uniform concentration of a salt, for example. The reservoirs 134, 136 may also include one or more reinforcements, such as a low basis weight non-woven scrim, for example, to provide shape retention to the hydrogels. The reservoirs 134, 136 each may have adhesive and cohesive properties that provide for releasable adherence to an applied area of a membrane (e.g., the skin of a patient). In various embodiments, the strength of an adhesive bond formed between portions of the assembly 100 and the application area or areas of the membrane is less than the strength of an adhesive bond formed between the membrane and the reservoirs 134, 136. These adhesive and cohesive properties of the reservoirs 134, 136 have the effect that when the assembly 100 is removed from an applied area of a membrane, a substantial amount of adhesive residue, for example, does not remain on the membrane. These properties also permit the reservoirs 134, 136 to remain substantially in electrical communication with their respective electrodes 104, 136 and the flexible backing 108 to remain substantially in surface contact with the printed electrode layer 102.

Portions of the assembly 100, as provided in accordance with embodiments of the present invention, may be structured to exhibit flexibility or low flexural rigidity in multiple directions along the structure of the device 100. Working against flexibility of the device 100, however, may be the construction of the comparatively stiffer electrode layer 102, which may include a material such as print-treated polyethylene terephthalate (PET), for example, as a substrate. PET is a relatively strong material exhibiting high tensile strength in both the machine and transverse directions and having a flexural rigidity, G=E*δⁿ, which is a function of modulus of elasticity (E) and a power of the thickness (δ) of the material. By way of a hypothetical counter-example, if a substance such as MYLAR™, for example, were to be used for both the electrode layer 102 and the flexible backing 108, at least two problems would be presented: (1) the assembly 100 would be too inflexible to fully or effectively adhere to a site of treatment on a membrane, and (2) upon removal from the membrane once treatment is completed, the assembly 100 would require a relatively high level of force, due to the strength of the flexible backing 108, to remove the assembly 100.

Embodiments of the present invention provide the flexible backing 108 around the periphery of the stiff electrode layer 102. In certain aspects, a relatively thin and highly compliant flexible backing composed of about 0.010 cm (0.004 inch) ethylene vinyl acetate (EVA), for example, may be used for the flexible backing 108. This configuration offers a flexible and compliant assembly 100 in multiple planar directions, permitting the assembly 100 to conform to the contour of a variety of membranes and surfaces. In addition, a pressure sensitive adhesive (e.g., polyisobutylene (PIB)) may be applied as the transfer adhesive layer 110 to mitigate a potential decrease in flexibility of the flexible backing 108. It can be seen that, in various embodiments, devices constructed in accordance with the present invention permit a degree of motion and flexure during treatment without disrupting the function of the assembly 100. The assembly 100 therefore exhibits low flexural rigidity in multiple directions, permitting conformability of the assembly 100 to a variety of membrane surface area configurations in a manner that is substantially independent of the chosen orientation of the assembly 100 during normal use. In various embodiments, a flexural rigidity of at least a portion of the flexible backing 108 is less than a flexural rigidity of at least a portion of the electrode layer 102.

In general, one advantage of the embodiments of the present invention is realized in minimization of the “footprint” of the assembly 100 when the assembly 100 is applied to a membrane to deliver a composition. As applied herein, the term “footprint” refers to the portion or portions of the assembly 100 that contact a membrane surface area (e.g., a patient's skin) during operation of the assembly 100. In certain aspects, the surface area of an assembly including the donor electrode 104 and the donor reservoir 134 may be structured to be greater than the surface area of an assembly including the return electrode 106 and the return reservoir 134 to limit the effect of the return assembly on the overall footprint of the assembly 100. In addition, the length of the distance 152 that provides separation between the anode 104 and cathode 106 may also impact the footprint. Furthermore, the size of the electrodes 104, 106 relative to their respective reservoirs 134, 136 may also affect the footprint of the assembly 100. In certain aspects, the reservoirs 134, 136 should be at least substantially the same size as their respective electrodes 104, 106.

It can be appreciated that the inventors have also recognized that once the surface area of the electrode layer 102 is fixed, including configuration of the anode 104 and cathode 106 separation distance 152, the assembly 100 should be sufficiently flexible and adherent for use on a membrane (e.g., a patient's skin). These objectives may depend on the peripheral area of the transfer adhesive layer 110 that surrounds the stiff electrode layer 102. In various embodiments, the width of the peripheral area of the transfer adhesive layer 110 adjacent to one or both of the anode 104 and cathode 106 may be provided as a minimum width 137 (as shown, for example, in FIG. 4). The minimum width 137 may be structured, in certain aspects, in the range of at least about 0.953 cm (0.375 inches). In turn, these objectives depend on the aggressiveness of the transfer adhesive layer 110 and the flexible backing 108, which is preferably flexible and compliant as a function of the strength (e.g., modulus of elasticity) and thickness of the flexible backing 108. Any sufficiently thin material may be flexible (such as ultra-thin PET, for example), but another problem arises in that the transfer adhesive layer 110 and the flexible backing 108 should be capable of removal from a membrane with minimum discomfort to a patient, for example. Consequently, a compliant (i.e., low strength) flexible backing 108 may be employed while maintaining adequate strength for treatments using the assembly 100.

In various example aspects of the structure of the present invention, the footprint area of the assembly 100 may be preferably in the range of about 3 cm² to 100 cm², more preferably in the range of about 5 cm² to 60 cm², and most preferably in the range of about 20 cm² to 30 cm. In addition, the total electrode 104, 106 area may be in the preferred range of about 2 cm² to 50 cm or more preferably in the range of about 3 cm to 30 cm² and most preferably in the range of about 4 cm² to 40 cm². In one operational example, the total contact area for the electrodes 104, 106 is about 6.3 cm² and the total reservoir 134, 136 contact area is about 7.5 cm². The ratio of the area of each reservoir 134, 136 to its corresponding electrode 104, 106 may be in the range of about 1.0 to 1.5. In other aspects, the flexible transfer adhesive 110 for the printed electrode layer 102 may have a thickness in the range of about 0.0038 cm (0.0015 inches) to about 0.013 cm (0.005 inches). The flexible backing 108 may be comprised of a suitable material such as EVA, polyolefins, polyethylene (PE, particularly low-density polyethylene (LDPE)), polyurethane (PU), and/or other similarly suitable materials.

In other example aspects of the structure of the present invention, the ratio of total electrode surface area to total footprint area may be in the range about 0.1 to 0.7, or preferably about 0.24. In certain aspects, the ratio of donor electrode 104 surface area to return electrode 106 surface area may be in the range of about 0.1 to 5.0, or preferably about 1.7. In still other aspects, the ratio of donor reservoir 134 thickness to return reservoir 136 thickness may be in the range of about 0.1 to 2.0, or more preferably about 1.0.

In various embodiments, the donor electrode reservoir 134, for example, may be loaded with an active ingredient from an electrode reservoir loading solution by placing an aliquot of the loading solution directly onto the hydrogel reservoir and permitting the loading solution to absorb and diffuse into the hydrogel over a period of time (this method of loading is shown in Platform II). FIG. 10 illustrates this method for loading of electrode reservoirs in which an aliquot of loading solution is placed on the hydrogel reservoir for absorption and diffusion into the reservoir. FIG. 10 is a schematic cross-sectional drawing of an anode electrode assembly 274 including an anode 280 and an anode trace 281 on a backing 288 and an anode reservoir 284 in contact with the anode 280. An aliquot of a loading solution 285, containing a composition to be loaded into the reservoir 284 is placed in contact with reservoir 284. Loading solution 285 is contacted with the reservoir 284 for a time period sufficient to permit a desired amount of the ingredients in loading solution 285 to absorb and diffuse into the gel reservoir 284. It can be appreciated that any suitable method or apparatus known to those in the art may be employed for loading the reservoir 284 with a composition.

In other embodiments of the present invention, at least one of the hydrogel reservoirs 134, 136 is positioned for electrical communication with at least a portion of at least one of the electrodes 104, 106. In various aspects, a surface area of at least one of the hydrogel reservoirs 134, 136 may be greater than or equal to a surface area of its corresponding electrode 104, 106. At least one of the hydrogel reservoirs 134, 136 may be loaded with a composition to provide a loaded hydrogel reservoir below an absorption saturation of the loaded hydrogel reservoir. In addition, at least one component of the assembly 100 in surface contact with, or in the vicinity of, the loaded hydrogel reservoir may have an aqueous absorption capacity less than an aqueous absorption capacity of the loaded hydrogel reservoir. In certain embodiments, a first kind of material comprising the unloaded hydrogel reservoir 134 in electrical communication with the anode electrode 104 is substantially identical to a second kind of material comprising the second unloaded hydrogel reservoir 136 in electrical communication with the cathode electrode 106.

In other embodiments of the present invention, a slit 202 may be formed in the flexible backing 108 in an area located between the anode 104 and the cathode 106 of the assembly 100. The slit 202 facilitates conformability of the assembly 100 to a membrane by dividing stress forces between the portion of the assembly including the anode and the portion of the assembly including the cathodes. In various embodiments, the electrode assembly 100 includes one or more non-adhesive tabs 206 and 208 that extend from the flexible backing 108 and to which no type of adhesive is applied. The non-adhesive tabs 206, 208 permit, for example, ready separation of the release cover 138 from its attachment to the electrode assembly 100. The non-adhesive tabs 206, 208 also may facilitate removal of the assembly 100 from a membrane (e.g., a patient's skin) on which the assembly 100 is positioned for use.

As described above, at least a portion of at least one of the anode electrode trace 112 and the cathode electrode trace 114 may be covered with an insulating dielectric coating 118 at portions along the traces 112, 114. The insulating dielectric coating 118 may be structured not to extend to cover completely the portion of the traces 112, 114 located at the tab end portion 116 of the assembly 100. This permits electrical contact between the traces 112, 114 and the electrical contacts of an interconnect device such as the flexible circuit connector 250B of the connector assembly 250. In various embodiments, the dielectric coating 118 may cover at least a portion of at least one of the anode 104/reservoir 134 assembly and/or the cathode 106/reservoir 136 assembly. In addition, the dielectric coating 118 may cover substantially all or at least a portion of a periphery of at least one of the electrodes 104, 106 and/or the traces 112, 114.

In various embodiments of the present invention, a gap 212 may be provided between a portion of the layer of transfer adhesive 110 nearest to the tab end portion 116 and a portion of the tab stiffener 124 nearest to the layer of transfer adhesive 110 to facilitate removal or attachment of the assembly 100 from/to a component of an electrically assisted delivery device such as the connector assembly 250, for example. In certain example embodiments, the gap 212 is at least about 1.27 cm (0.5 inches) in width. The gap 212 provides a tactile sensation aid such as for manual insertion, for example, of the assembly 100 into the flexible circuit connector 250B of the connector assembly 250. The gap 212 may also provide relief from stress caused by relative movement between the assembly 100 and other components of a delivery device (e.g., the connector assembly 250) during adhesion and use of the assembly 100 on a membrane.

In addition, at least one tactile feedback notch 214 and one or more wings 216, 218 may be formed in or extend from the tab end 116 of the electrode assembly 100. The feedback notch 214 and/or the wings 216, 218 may be considered tactile sensation aids that facilitate insertion or removal of the tab end 116 into/from a component of an electrically assisted delivery device such as, for example, to establish an operative association with the flexible circuit connector 250B of the connector assembly 250.

FIGS. 6B and 6C each show the layering of elements of the electrode assembly 100 as shown in FIG. 6. In FIGS. 6B and 6C, it can be seen that the thickness of layers is not to scale and adhesive layers are omitted for purposes of illustration. FIG. 6B shows a cross section of the anode electrode 104/reservoir 134 assembly and the cathode electrode 106/reservoir 136 assembly. The anode 104 and the cathode 106 are shown layered on the printed electrode layer 102. The anode reservoir 134 and the cathode reservoir 136 are shown layered on the anode 104 and the cathode 106, respectively. FIG. 6C is a cross-sectional view through the anode 104, the anode trace 112, and the anode reservoir 134. The anode 104, the anode trace 112 and a sensor trace 130 are layered upon the electrode layer 102. The anode reservoir 134 is shown in electrical communication with the anode 104. The tab stiffener 124, which may be composed of an acrylic material, for example, is shown attached to the tab end 116 of the assembly 100. In addition, the sensor trace 130 may be located at the tab end 116 of the electrode assembly 100.

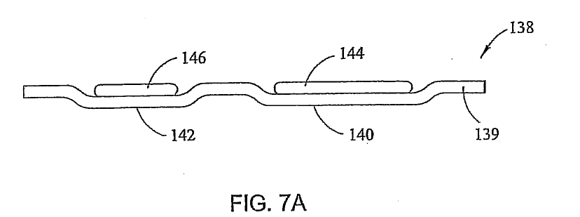

In other embodiments of the present invention, FIGS. 7 and 7A show schematically the release cover 138 structured for use with various devices, electrode assemblies and/or systems of the present invention. The release cover 138 includes a release cover backing 139, which includes an anode absorbent well 140 and a cathode absorbent well 142. In various exemplary aspects, a nonwoven anode absorbent pad may be contained within the anode absorbent well 140 as the transfer absorbent 144, and a nonwoven cathode absorbent pad may be contained within the cathode absorbent well 142 as the transfer absorbent 146. In use, the release cover 138 is attached to the electrode assembly 100 so that the anode absorbent pad 144 and the cathode absorbent pad 146 substantially cover the anode reservoir 134 and the cathode reservoir 136, respectively. The anode absorbent pad 144 and the cathode absorbent pad 146 may each be slightly larger than their corresponding anode reservoir 134 or cathode reservoir 136 to cover and protect the reservoirs 134, 136. The anode absorbent pad 144 and the cathode absorbent pad 146 may also be slightly smaller than the anode absorbent well 140 and the cathode absorbent well 142, respectively. In various embodiments, one or more indicia 220 (e.g., a “+” symbol as shown) may be formed on at least a portion of the flexible backing 108 of the assembly 100 adjacent to the anode well 140 and/or the donor well 142. It can be appreciated that the indicia 220 may promote correct orientation and use of the assembly 100 during performance of an iontophoretic procedure, for example.

The anode absorbent pad 144 and the cathode absorbent pad 146 may be attached to the backing 139 of the release cover 138 by one or more ultrasonic spot welds such as welds 222, 224, 226, for example, as shown in FIG. 7. The welds 222, 224, 226 may be substantially uniformly distributed in areas of connection between the non-woven fabric pads 144, 146 and the wells 140, 142, respectively.

To facilitate removal of the release cover 138 from the electrode assembly 100, portions of the backing 139 in communication with the transfer adhesive 110 when the release cover 138 is attached to the electrode assembly 100 may be treated with a release coating, such as a silicone coating, for example.

### Packaging of Iontophoretic Device

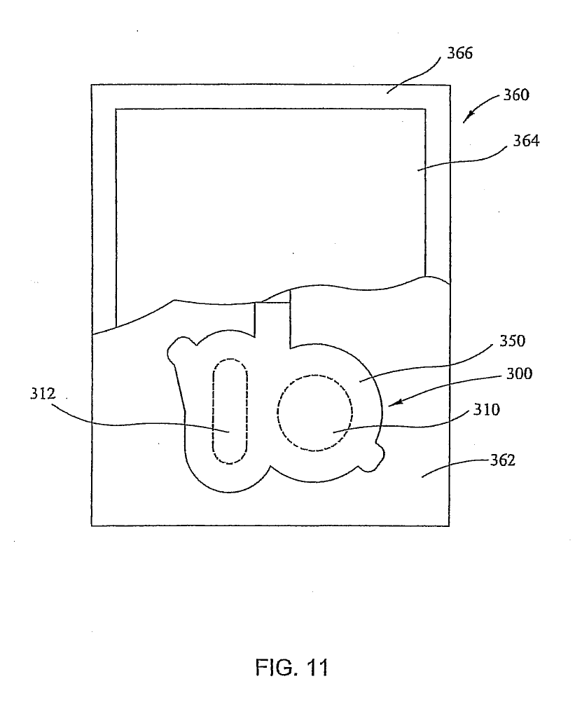

FIG. 11 is a breakaway schematic representation of the electrode assembly 300 within a hermetically sealed packaging 360. Packaged electrode assembly 300 is shown with release liner 350 in place and anode 310 and cathode 312 are shown in phantom for reference. Hermetically sealed packaging 360 is a container that is formed from a first sheet 362 and a second sheet 364, which are sealed along seam 366. Hermetically sealed packaging 360 can be of any suitable composition and configuration, so long as, when sealed, substantially prevents permeation of any fluid or gas including, for example, permeation of oxygen into the packaging 360 and/or the loss of water from the packaging 360 after the electrode assembly 300 is sealed inside the hermetically sealed packaging 360.

In use, sheets 362 and 364 are sealed together to form a pouch after electrode assembly 300 is placed on one of sheets 362 and 364. Other techniques well-known to those skilled in the art of packaging may be used to form a hermetically sealed package with an inert atmosphere. In one embodiment, the moles of oxygen in the inert gas in the sealed pouch is limited, by controlling the oxygen concentration in the inert gas and by minimizing the internal volume, or headspace, of the package, to be slightly less than the amount of sodium metabisulfite in the epinephrine-containing reservoir needed to react with all oxygen in the package. Electrode assembly 300 is then inserted between sheets 362 and 364, an inert gas, such as nitrogen is introduced into the pouch to substantially purge air from the pouch, and the hermetically sealed packaging 360 is then sealed. The hermetically sealed packaging 360 may be sealed by adhesive, by heat lamination or by any method know to those skilled in the art of packaging devices such as electrode-assembly 300. It should be noted that sheets 362 and 364 may be formed from a single sheet of material that is folded onto itself, with one side of hermetically sealed packaging 360 being a fold in the combined sheet, rather than a seal. In other embodiments, the sheets 362, 364 may be formed from individual sheets that are laminated together, for example, to form a package. Other container configurations would be equally suited for storage of electrode-assembly, so long as the container is hermetically sealed.

Sheets 362 and 364, and in general, hermetically sealed packaging 360 may be made form a variety of materials. In one embodiment, the materials used to form hermetically sealed packaging 360 has a laminate structure comprising 48 gauge PET/Primer/15 lb LDPE (low density polyethylene)/0.025 mm (1.0 mil) aluminum foil adhesive/48 gauge PET/10 lb LDPE chevron pouch 0.05 mm (2 mil) peelable layer. Laminates of this type (foil, olefinic films and binding adhesives) form strong and channel-free seals and are essentially pinhole-free, assuring essentially zero transfer of gases and water vapor for storage periods up to and exceeding 24 months. Other suitable barrier materials to limit transport of oxygen, nitrogen and water vapor for periods of greater than 24 months are well-known to those of skill in the art, and include, without limitation, aluminum foil laminations, such as the INTEGRA® products commercially available from Rexam Medical Packaging of Mundelein, Ill.

It can be appreciated that any of the assemblies, devices, systems, or other apparatuses described herein may be, where structurally suitable, included within hermetically sealed packaging as described above.

### Protocols for Loading Reservoirs

In use, electrode reservoirs described herein can be loaded with an active ingredient from an electrode reservoir loading solution according to any protocol suitable for absorbing and diffusing ingredients into a hydrogel. Two protocols for loading a hydrogel include, without limitation, 1) placing the hydrogel in contact with an absorbent pad material, such as a nonwoven material, into which a loading solution containing the ingredients is absorbed and 2) placing an aliquot of the loading solution directly onto the hydrogel and permitting the loading solution to absorb and diffuse into the hydrogel over a period of time.

In the first protocol, the loading solution containing ingredients to be absorbed and diffused into the respective anode reservoir 134 and cathode reservoir 136 are first absorbed into the nonwoven anode absorbent pad 144 and nonwoven cathode absorbent pad 146, respectively. When a release cover thus loaded is connected to electrode assembly 100, the ingredients therein desorb and diffuse from the absorbent pads 144 and 146 and into the respective reservoir. In this case, absorption and diffusion from the reservoir cover into the reservoirs has a transfer efficiency of about 95%, requiring that about a 5% excess of loading solution be absorbed into the absorbent pads. Despite this incomplete transfer, the benefits of this loading process, as compared to placing a droplet of loading solution onto the reservoirs and waiting between about 16 and 24 hours or so for the droplet to immobilize and absorb, are great because once the release cover is laminated onto the electrode assembly, the assembly can be moved immediately for further processing and placed in inventory. There is no requirement that the assembly is kept flat and immobile while awaiting completion of absorption and/or diffusion.

The transfer absorbents 144 and 146 are typically a nonwoven material. However, other absorbents may be used, including woven fabrics, such as gauze pads, and absorbent polymeric compositions such as rigid or semi-rigid open cell foams. In the particular embodiments described herein, the efficiency of transfer of loading solution from the absorbent pads of the release cover to the reservoirs is about 95%. It would be appreciated by those skilled in the art of the present invention that this transfer efficiency will vary depending on the composition of the absorbent pads and the reservoirs as well as additional physical factors including, without limitation, the size, shape and thickness of the reservoirs and absorbent pads and the degree of compression of the absorbent pad and reservoir when the release cover is affixed to the electrode assembly. The transfer efficiency for any given release cover-electrode assembly combination can be readily determined empirically and, therefore, the amount of loading solution needed to fully load the reservoirs to their desired drug content can be readily determined to target specifications.

As discussed above, FIG. 10 illustrates the second protocol for loading of electrode reservoirs in which an aliquot of loading solution is placed on the hydrogel reservoir for absorption and diffusion into the reservoir. The transfer absorbents 144, 146 typically are not included in the release cover for electrode assemblies having reservoirs loaded by this method.

In various embodiments, the electrode assembly 100 is manufactured, in pertinent part, by the following steps. First, electrodes 104 and 106 and traces 112, 114 and 130 are printed onto a polymeric backing, such as ink-printable treated PET film, for example, or another suitably semi-rigid dimensionally stable material. The dielectric layer 118 may then be deposited onto the appropriate portions of traces 112 and 114 that are not intended to electrically contact the electrode reservoirs and contacts of an interconnect between the electrode assembly and a power supply/controller, for example. The polymeric backing onto which the electrodes are printed is then laminated to the flexible backing 108. The anode reservoir 134 and cathode reservoir 136 are then positioned onto the electrodes 104 and 106, respectively. In the assembly of the release cover 138 under the first protocol, the transfer absorbents 144 and 146 are ultrasonically spot welded within wells 140 and 142 and are loaded with an appropriate loading solution for absorption and/or diffusion into the anode and/or cathode reservoirs 134 and 136. An excess of about 5% loading solution (over the amount needed to absorb and diffuse into the hydrogel) typically is added to the reservoir covers due to in the about 95% transfer efficiency of the loading process, resulting in some of the loading solution remaining in the absorbent reservoir covers.

Once assembled and loaded with loading solution, the release cover is positioned on the electrode assembly 100 with the loaded transfer absorbents 144 and 146 in surface contact with anode and cathode reservoirs 134 and 136, respectively. Over a time period, typically at least about 24 hours, substantial portions (about 95%) of the loading solutions are absorbed and diffused into the hydrogel reservoirs. The completed assembly is then packaged in an inert gas environment and hermetically sealed.

### Methods of Use

In one method of use, the release cover 138 is removed from the electrode assembly 100, and the electrode assembly 100 is placed on a patient's skin at a suitable location. After the electrode assembly 100 is placed on the skin, it is inserted into a suitable interconnect, such as a component of the connector assembly 250, for example. An electric potential is applied according to any profile and by any means for electrically assisted drug delivery known in the art. Examples of power supplies and controllers for electrically assisted drug delivery are well known in the art, such as those described in U.S. Pat. Nos. 6,018,680 and 5,857,994, among others. Ultimately, the optimal current density, drug concentration and duration of the electric current (including profiles with time) and/or electric potential is determined and/or verified experimentally for any given electrode/electrode reservoir combination.

The electrodes described herein are standard Ag or Ag/AgCl electrodes and can be prepared in any manner according to standard methods in such a ratio of Ag to AgCl (if initially present), thickness and pattern, such that each electrode will support the electrochemistry for the desired duration of treatment. Typically, as is common in preparation of disposable iontophoresis electrodes, the electrodes and electrode traces are prepared by printing Ag/AgCl ink in a desired pattern on a stiff polymeric backing, for example print treated 0.005 cm (0.002 inch) PET film, by standard lithographic methods, such as by rotogravure. Ag/AgCl ink is commercially available from E.I. DuPont de Nemours and Company, for example and without limitation, DuPont Product Id. Number 5279. The dielectric also may be applied to the electrode traces by standard methods. As with the electrode, dielectric ink may be applied in a desired pattern over the electrodes and electrode traces by standard printing methods, for instance by rotogravure.

The pressure-sensitive adhesive (PSA) and transfer adhesives may be any pharmaceutically acceptable adhesive suitable for the desired purpose. In the case of the pressure-sensitive adhesive, the adhesive may be any acceptable adhesive useful for affixing an electrode assembly to a patient's skin or other membrane. For example, the adhesive may be polyisobutylene (PIB) adhesive. The transfer adhesive, used to attach different layers of the electrode assembly to one another, also may be any pharmaceutically acceptable adhesive suitable for that purpose, such as PIB adhesive. For assembly of the electrodes described herein, the PSA typically is provided pre-coated on the backing material with a silicone-coated release liner attached thereto to facilitate cutting and handling of the material. Transfer adhesive typically is provided between two layers of silicone-coated release liner to facilitate precise cutting, handling and alignment on the electrode assembly.

The anode and cathode reservoirs described herein may comprise a hydrogel. The hydrogel typically is hydrophilic and may have varying degrees of cross-linking and water content, as is practicable. A hydrogel as described herein may be any pharmaceutically and cosmetically acceptable absorbent material into which a loading solution and ingredients therein can be absorbed, diffused or otherwise incorporated and that is suitable for electrically assisted drug delivery. Suitable polymeric compositions useful in forming the hydrogel are known in the art and include, without limitation, polyvinylpyrrolidone (PVP), polyethyleneoxide, polyacrylamide, polyacrylonitrile and polyvinyl alcohols. In one non-limiting embodiment, the hydrogel may comprise from about 15% to about 17% by weight polyvinyl pyrrolidone. The reservoirs may contain additional materials such as, without limitation: preservatives, such as Phenonip Antimicrobial, available commercially from Clariant Corporation of Mount Holly, N.C.; antioxidants, such as sodium metabisulfite; chelating agents, such as ethylenediaminetetraacetic acid (EDTA); and humectants. A typical unloaded reservoir contains preservatives and salt. As used herein in reference to the water component of the electrode reservoirs, the water is purified and preferably meets the standard for purified water in the USP XXIV.

As discussed above, the hydrogel has sufficient internal strength and cohesive structure to substantially hold its shape during processing, forming and for its intended use and leave essentially no residue when the electrode is removed after use. As such, the cohesive strength of the hydrogel and the adhesive strength between the hydrogel and the electrode are each greater than the adhesive strength of the bonding between the hydrogel and the membrane (for instance skin) to which the electrode assembly is affixed in use. In one non-limiting embodiment, the hydrogel may have a thickness from about 0.089 cm (0.035 inch) to 0.114 cm (0.045 inch).

The unloaded donor (anode) reservoir also includes a salt, preferably a fully ionized salt, for instance a halide salt such as sodium chloride in a concentration of from about 0.001 wt. % to about 0.1 wt. %, preferably from about 0.01 wt. % to about 0.09 wt. % and most preferred about 0.06 wt. %. The salt content is sufficient to prevent electrode corrosion during manufacture and shelf-storage of the electrode assembly. These amounts may vary for other salts in a substantially proportional manner depending on a number of factors, including the molecular weight and valence of the ionic constituents of each given salt in relation to the molecular weight and valence of sodium chloride. Other salts, such as organic salts, are useful in ameliorating the corrosive effects of certain drug salts. Typically the best salt for any ionic drug will contain an ion that is the same as the counter ion of the drug. For instance, acetates would be preferred when the drug is an acetate form. However, the aim is to prevent corrosion of the electrodes.

Lidocaine HCl and epinephrine bitartrate are used in the examples below to elicit a desired pharmacological response. If the counterion of lidocaine is not chloride, though chloride ions may be useful to prevent electrode corrosion, a corrosion-inhibiting amount of that other counterion may be present in the unloaded reservoir in addition to, or in lieu of the chloride ions to prevent corrosion of the electrode. If more than one counterion is present, such as in the case where more than one drug is loaded and each drug has a different counterion, it may be preferable to include sufficient amounts of both counterions in the reservoir to prevent electrode corrosion. It should be noted that in the examples provided below, the amount of epinephrine bitartrate loaded into the gel is not sufficient to cause corrosion.

The return (cathode) reservoir may be a hydrogel with the same or different polymeric structure and typically contains a salt such as sodium chloride, a preservative and, optionally, a humectant. Depending upon the ultimate manufacturing process, certain ingredients may be added during cross-linking of the hydrogel reservoir, while others may be loaded with the active ingredients. Nevertheless, it should be recognized that irrespective of the sequence of addition of ingredients, the salt must be present in the reservoir adhering to the electrode and substantially evenly distributed therethrough prior to the loading of the active ingredient(s) or other ingredient to limit the formation of concentration cells.

### Stability

As briefly mentioned above, “stability” may refer to a variety of qualities of the reservoir-electrode. Drug or pharmaceutical stability is one parameter. For instance, epinephrine typically is very unstable. Therefore, an iontophoretic electrode assembly might be considered stable for the time period that useful quantities of epinephrine remain available for delivery. Similarly, if lidocaine is considered, the electrode assembly remains stable for the time period that useful quantities of lidocaine remain available for delivery.

Described with specificity herein is an embodiment of an iontophoretic system for delivery of the topical anesthetic lidocaine with the vasoconstrictor epinephrine, more specifically lidocaine HCl and epinephrine bitartrate as shown in the Examples. The particular amounts of epinephrine and lidocaine shown in the Examples are selected to produce effective local anesthesia. According to one non-limiting embodiment, the reservoir may comprise lidocaine HCl and epinephrine bitartrate in about a 50-1000:1 mass ratio. In another non-limiting embodiment, the reservoir may comprise lidocaine HCl and epinephrine bitartrate in about a 75-125:1 mass ratio. Variations in the relative concentration and/or mass of lidocaine and/or epinephrine, as well as variations in reservoir volume, reservoir composition, reservoir skin contact surface area, electrode size and composition and electrical current profile, among other parameters, could result in changes in the optimal concentrations of lidocaine and/or epinephrine in the gel reservoir. A person of skill in the art would be able to adjust the relative amounts of ingredients to achieve the same results in a system in which any physical, electrical or chemical parameter differs from those disclosed herein.

For most, if not all applications, epinephrine stability should not be dependent upon epinephrine concentration within a range that can be extrapolated from the data provided herein. A useful range of epinephrine is, therefore, from about 0.01 mg/ml to about 2.0 mg/ml.

Although lidocaine is a common topical anesthetic, other useful topical (surface and/or infiltration) anesthetics may be used in the described system. These anesthetics include, without limitation, salts of: amide type anesthetics, such as bupivacaine, butanilicaine, carticaine, cinchocaine/dibucaine, clibucaine, ethyl parapiperidino acetylaminobenzoate, etidocaine, lidocaine, mepivicaine, oxethazaine, prilocaine, ropivicaine, tolycaine, trimecaine and vadocaine; ester type anesthetics, including esters of benzoic acid such as amylocaine, cocaine and propanocaine, esters of metaaminobenzoic acid such as clormecaine and proxymetacaine, esters of paraaminobenzoic acid (PABA) such as, amethocaine (tetracaine), benzocaine, butacaine, butoxycaine, butyl aminobenzoate, chloroprocaine, oxybuprocaine, parethoxycaine, procaine, propoxycaine and tricaine; and miscellaneous anesthetics, such as, bucricaine, dimethisoquin, diperodon, dyclocaine, ketocaine, myrtecaine, octacaine, pramoxine and propipocaine.

Of the topical anesthetics, salts of bupivacaine, butacaine, chloroprocaine, cinchocaine, etidocaine, mepivacaine, prilocaine, procaine, ropivacaine and tetracaine (amethocaine) might be considered by some to be more clinically relevant than other anesthetics listed above, though not necessarily more effective. Certain other features of each of the compounds listed above may make any particular compound more or less suited to iontophoretic delivery as described herein. For example, use of cocaine may be contra-indicated because of its cardiovascular side effects. Bupivacaine, butacaine, chloroprocaine, cinchocaine, etidocaine, mepivacaine, prilocaine, procaine, ropivacaine and tetracaine (amethocaine) may be preferred as substitute for lidocaine because the all have similar pKs of about 8 or greater than 8, meaning they will ionize under the same conditions as lidocaine. Iontophoresis in vitro across human skin has shown that bupivacaine and mepivacaine show a similar cumulative delivery as lidocaine, while etidocaine, prilocaine and procaine have shown slightly greater delivery. Chloroprocaine, procaine and prilocaine have similar relatively short duration effects (less than 2 hr) whereas bupivicaine, etidocaine, and mepivacaine have effects lasting 3-4 hr. These times are approximately doubled when epinephrine is used in conjunction with these anesthetics. The duration of the action of the local anesthetic is dependent upon the time for which it is in contact with the sensory nerves. This duration of effect will depend on the physiochemical and pharmacokinetic properties of the drug. Hence, any procedure that can prolong contact between the therapeutic agent and the nerve, such as co-delivery of a vasoconstrictor with the anesthetic, will extend the duration of action.

Another factor that should be considered is that ester-based anesthetics based on PABA are associated with a greater risk of provoking an allergic reaction because these esters are metabolized by plasma cholinesterase to yield PABA, a known allergen. For this reason, amide anesthetics might be preferred and molecules such as chloroprocaine, and procaine would not be viewed as first-line replacements for lidocaine. Because bupivacaine, etidocaine, mepivacaine, ropivicaine and prilocaine are amide anesthetics with similar physiochemical properties and clinical effects as lidocaine, they may be preferred by some as substitutes for lidocaine. A secondary issue with prilocaine is that although it is generally considered to be the safest of the amide anesthetics, one of its metabolites (o-toluidine) has been associated with increased risk of methemoglobinemia and cyanosis as compared to the other amide anesthetics.

Each of the anesthetics listed above have varying degrees of vasoconstrictor activity. Therefore, optimal concentrations of the anesthetic and the vasoconstrictor will vary depending on the selected local analgesic. However, for each local anesthetic, optimal effective concentration ranges can be readily determined empirically by functional testing. As used herein, the terms “anesthetic” and “anesthesia” refer to a loss of sensation, and are synonymous with “analgesics” and “analgesia” in that a patient's state of consciousness is not considered when referring to local effects of use of the described iontophoretic device, even though some of the drugs mentioned herein may be better classified as “analgesics” or “anesthetics” in their systemic use. Sodium metabisulfite may be added to the donor reservoir to scavenge oxygen. The amount of sodium metabisulfite added is not substantially in excess of the amount needed to scavenge all oxygen from the packaged reservoir for a given time period to minimize the formation of the adduct epinephrine sulfonic acid, and/or other decomposition products. For example, the donor hydrogel may contain less than about 110%, for example about 101%, of the amount of sodium metabisulfite equal to a minimal amount of sodium metabisulfite needed to scavenge substantially all oxygen in the packaged donor hydrogel. The amount of sodium metabisulfite needed to scavenge oxygen in the packaged donor hydrogel for any given amount of time can be calculated from the amount of oxygen present within the package in which the donor hydrogel is hermetically sealed. Alternately, the optimal amount of sodium metabisulfite can be titrated by determining the amount of sodium metabisulfite at which production of the oxidation products of epinephrine due to its reaction with oxygen, such as adrenolone or adrenochrome, and epinephrine sulfonic acid, essentially stops.

### Measurements of Dermal Analgesia

Aesthesiometers are used to test the threshold for the tactile receptors in the skin. They are widely used in hand surgery and rehabilitation to detect and monitor peripheral nerve function or results of nerve repair. They are also used to objectively determine touch thresholds, screening for peripheral nerve impairment, determining spatial extent and degree of nerve impairment, and detecting changes in neurological status. For example, aesthesiometers can be used to determine the location and delineation of areas of analgesia, or absence of pain and touch sensitivity, as well as areas of hyposthesia, that is reduced pain or touch sensitivity, of the skin of a person, for example, as is shown below. A very common type of aesthesiometer is a filament aesthesiometer in which a filament is pressed perpendicularly to the skin and the applied pressure is measured to determine tactile thresholds. Other aesthesiometers apply pressure in different ways, such as air pressure to measure tactile thresholds.

Around 1900, Max von Frey discovered that horse hairs tended to apply a single downward force that was not proportional to bending in that horse hairs could be used to measure anesthesia. In contrast, for the common spring, the downward force is directly proportional to the bending. Modern filament aesthesiometers use monofilaments, such as nylon monofilaments rather than horse hair. Nylon monofilament was not invented until WWII. Sidney Weinstein immediately thereafter employed the nylon monofilament to produce a set of 20 diameter-varying and length-constant monofilaments. These monofilaments produce a characteristic force perpendicular to the contacting surface. The characteristic forces for his set of monofilaments were published, and that set of nylon monofilaments on plexiglass handles is known today as the Semmes-Weinstein Aesthesiometer (SWA). “Aesthesiometer filaments,” collectively refer to any filament, such as, without limitation, horse hair or nylon monofilament, used in an aesthesiometer.

Aesthesiometer filaments will produce varying sensations of touch when applied to the skin. By applying an increasing axial force along the filament, with one end of the filament engaged with and perpendicular to the patient's skin, the filament will apply an increasing force on the patient's skin. As the monofilaments are placed on the skin, they begin to bend. This force can be so small that tactile receptors cannot sense it. When the column buckling stress of the filament is reached, the filament will bend sideways in an arch as the force and pressure applied to the patient by the filament decreases from a predetermined maximum value.

By standardizing the length, diameter and modulus of the filament, a standardized present maximum force can be repeatedly applied to a patient at the point where the filament initiates buckling. Each monofilament number corresponds to level of force provided by that monofilament. The common monofilament is a single strand of nylon, which has the property of producing a characteristic downward force when buckled on a surface. The downward force does not depend on the degree of bend of the monofilament. Once in contact with the skin, the monofilament starts to bend and reaches a force maximum that is not exceeded with further bending. The actual force varies around the characteristic force for that monofilament. Equations predict the characteristic force from the diameter and the length of the monofilament.

In embodiments of the present invention, aesthesiometer measurements were used to determine the level of analgesia achieved by the described iontophoretic devices. In particular embodiments of the invention, an amount of a vasoconstrictor and an anesthetic are pre-loaded in an iontophoretic device. Drug delivery is then electrically assisted for a period of time, producing at least a 50% reduction of dermal sensitivity to an applied force as measured by a filament aesthesiometer and producing a hedonic score of greater than about −1.5 on a visual analogue scale ranging from −10 to 10. In these embodiments, the vasoconstrictor is delivered in an amount that will not result in skin necrosis, i.e., will not necrotize the skin.

In particular embodiments, these anesthetics include, without limitation, salts of: amide type anesthetics, such as bupivacaine, butanilicaine, carticaine, cinchocaine/dibucaine, clibucaine, ethyl parapiperidino acetylaminobenzoate, etidocaine, lidocaine, mepivicaine, oxethazaine, prilocaine, ropivicaine, tolycaine, trimecaine and vadocaine; ester type anesthetics, including esters of benzoic acid such as amylocaine, cocaine and propanocaine, esters of metaaminobenzoic acid such as clormecaine and proxymetacaine, esters of paraaminobenzoic acid (PABA) such as, amethocaine (tetracaine), benzocaine, butacaine, butoxycaine, butyl aminobenzoate, chloroprocaine, oxybuprocaine, parethoxycaine, procaine, propoxycaine and tricaine; and miscellaneous anesthetics, such as, bucricaine, dimethisoquin, diperodon, dyclocaine, ketocaine, myrtecaine, octacaine, pramoxine and propipocaine. In other embodiments, the amount of anesthetic used ranges from about 2% to 30% weight of the loading solution, preferably about 10% weight of the loading solution.

In yet further embodiments, examples of vasoconstrictors include, but are not limited to, epinephrine, norepinephrine, and phenylephrine. In certain embodiments, the vasoconstrictor may be a salt, such as for example, a bitartrate salt. In other embodiments, the vasoconstrictor may be a free base. In other particular embodiments, the amount of vasoconstrictor used ranges from 0.005% to 0.3% weight of the reservoir. In other particular embodiments, the amount of vasoconstrictor used ranges from 0.01% to 0.3% weight of the reservoir.

In particular embodiments, the period of time of electrically driven delivery ranges from 1 to 30 minutes, more preferably from 5 to 20 minutes. In one embodiment, the period of time of electrically driven delivery is about 10 minutes. In another embodiment, the period of time of electrically driven delivery is from 2 to 10 minutes. In yet other embodiments, the electrical assistance is provided by using current densities ranging from 0.1 to 6.0 mA·min/cm², more preferred 0.1 to 4.2 mA·min/cm², and most preferably between and including 2.4 to 3.4 mA·min/cm².

Aesthesiometer measurements were taken using a standard monofilament aesthesiometer. The invention is no way limited by use of this particular aesthesiometer and any of the commercially available aesthesiometers may be used. In particular embodiments, baseline aesthesiometer measurements were taken prior to applying an iontophoretic device to the skin of the subject. In yet further embodiments, aesthesiometer measurements were taken immediately post-treatment with an iontophoretic device and again minutes (ranging from, but not limited to, 20-30 minutes) later to determine the effectiveness of the iontophoretic device in producing localized analgesia. Monofilament fibers ranging from 1.65 to 6.65 gauge were used. The difference in detectable gauge size (detecting at least 3 of 5 touches) between the baseline measurement and measurement immediately after iontophoresis (DELTA0) and between baseline measurement and measurement 20 minutes after iontophoresis (DELTAI20) was assessed. In particular embodiments, subjects were tested when treated with 10% weight of lidocaine in the reservoir with varying amounts of epinephrine and varying charge densities. In yet further embodiments, the electrically assisted drug delivery system produced at least about a 50% reduction of dermal sensitivity to an applied force and producing a von Frey score DELTAI0 of at least 1.15. Use of the von Frey scale with the particular dosage of 10% lidocaine is in no way limiting regarding the use of this scale or the amount of lidocaine.

In yet further embodiments, degree of clinical analgesia was also determined by cannulation pain using a visual analogue scale (VAS), wherein pain was assessed on a scale from 0-100 mm. In this embodiment, pain associated with insertion of a catheter into a vein selected on the dorsum of the hand was measured with and without treatment according to the various non-limiting embodiments of the present disclosure. In the instant case, 0 mm represents no pain and 100 mm represents very severe pain. In particular embodiments, subjects were tested using this scale when treated with 10% weight of lidocaine in the reservoir with varying amounts of epinephrine and varying charge densities. In yet further embodiments, the electrically assisted drug delivery system produced at least about a 50% reduction of dermal sensitivity to an applied force and producing a cannulation pain score of less than about 51 mm on the VAS scale. Use of the VAS cannulation pain scale with the particular dosage of 10% lidocaine is in no way limiting regarding the use of this scale or the amount of lidocaine.

In other embodiments, the sensation of iontophoresis, i.e., the sensation associated with the application of iontophoresis using iontophoretic devices according to the various non-limiting embodiments of the present disclosure, was determined on a Sensation Intensity Scale (SIS), which ranged from no sensation to extremely intense sensation on a scale of 1 to 10, respectively. In particular embodiments, subjects were tested using this scale when treated with 10% weight of lidocaine in the reservoir with varying amounts of epinephrine and varying current densities. In yet further embodiments, the electrically assisted drug delivery system produced at least about a 50% reduction of dermal sensitivity to an applied force and producing a SIS score of less than about 7.4. Use of a SIS with the particular dosage of 10% lidocaine is in no way limiting regarding the use of this scale or the amount of lidocaine.

In yet further embodiments, the sensation of iontophoresis was determined on a Hedonic scale, which ranged from unpleasantness to pleasantness on a scale of −10 to 10. In particular embodiments, subjects were tested using this scale when treated with 10% weight of lidocaine in the reservoir with varying amounts of epinephrine and varying charge densities. In yet further embodiments, the electrically assisted drug delivery system produced at least about a 50% reduction of dermal sensitivity to an applied force and producing a hedonic score of greater than about −1.5. Use of the Hedonic scale with the particular dosage of 10% lidocaine is in no way limiting regarding the use of this scale or the amount of lidocaine.

In still further non-limiting embodiments, the erythema and edema Draize scores of the anode and cathode application sites was determined at three different times; immediately post-iontophoresis, 1 hour post-iontophoresis, and about 24 hours post-iontophoresis (see, Draize, J. H., et al., “Therapeutic Methods for the Study of Irritation and Toxicity,” J. M. Exp. (1944), 377-390). A scale of 0 to 4 was used to evaluate the severity of the erythema (redness) and edema (swelling), where 0 represents no erythema or no edema, respectively, and 4 represents severe erythema (beet redness to slight eschar formation) or severe edema (swelling raised more than 1 mm and beyond exposed area), respectively. In particular embodiments, subjects were tested using this scale when treated with 10% weight of lidocaine in the reservoir with varying amounts of epinephrine and varying charge densities. In yet further embodiments, the electrically assisted drug delivery system produced at least a Draize score for erythema of less than about 2 (well defined erythema) at either the anode or cathode application site and a Draize score for edema of less than about 1 (very slight edema—barely perceptible) at either the anode or cathode application site. The Draize scores for both erythema and edema resolved in less than about 48 hours. Use of Draize scores for erythema or edema with the particular dosage of 10% lidocaine is in no way limiting regarding the use of this scale or the amount of lidocaine.

### Patch Fabrication Platform I—Pre-Loaded Integrated Patch

FIGS. 2 through 11 illustrate various aspects of an integrated electrode assembly 100 structured for use with an electrically assisted delivery device, for example, for delivery of a composition through a membrane. Patch Fabrication Platform I corresponds to an integrated patch as described by FIGS. 2 through 11, except for FIG. 10. The integration patch of Patch Fabrication Platform I is pre-loaded with medicament, as disclosed above. A description of the pre-loaded integrated electrode assembly and the associated electrically assisted delivery device is found in co-pending application Ser. No. 10/820,346 filed Apr. 7, 2004, which device is referred to herein as the “Electrotransport device” and when used together with the drug to be delivered, “Electrotransport System”, as disclosed under the section headed by Platform I, above.

### Patch Fabrication Platform II—Droplet Loaded Integrated Patch

In another embodiment, unloaded gel reservoirs within an integrated patch assembly were prepared using PVP, phenonip, NaCl, and purified water. The unloaded anode gel reservoirs were placed on Ag/AgCl anodes and 0.32 mL aliquots of loading Solution were placed on the reservoirs and were permitted to absorb and diffuse into the reservoir.

### Patch Fabrication Platform III—Pre-loaded Split Patch

The following components were assembled to prepare an electrode assembly, essentially as shown in the figures discussed above, for delivery of lidocaine and epinephrine by iontophoresis. Patch system used in von Frey study were composed of 2 patches, an active patch (the anode) which contained lidocaine HCl, and in some cases contained epinephrine, and a return patch (the cathode) which contained only saline. Each patch was constructed of a Ag/AgCl electrode laminate, a polyvinylpyrrolidone (PVP) hydrogel reservoir, a polyethylene/acrylic adhesive laminate, and a siliconized release liner. The electrode material used for the construction of the anodes/cathodes was a Ag/AgCl printed ink material on a polyester substrate and did not exceed 5 cm².

Each drug patch was 5 cm² in active surface area with a 0.10 cm (0.04 inch) thick reservoir composed of irradiation crosslinked PVP hydrogel at a composition of 24%±1%. These hydrogels had an equilibrium capacity of greater than 2.5 (wt. of loaded hydrogel/wt. of unloaded hydrogel, g/g). The 5 cm² hydrogels were laminated to silver/silver chloride ink printed electrodes. The anodes were loaded with 0.32 mL (50% by weight of the unloaded hydrogel) of lidocaine HCl and epinephrine loading solution to attain the desired compositions. For example, a 10% lidocaine HCl, 0.1% epinephrine anode would have 30% lidocaine HCl and 0.3% epinephrine in a loading solution. A 10% lidocaine HCl, 0.01% epinephrine anode would have 30% lidocaine HCl and 0.03% epinephrine in a loading solution. Each electrode was covered with a siliconized PET release cover. The cathode always contained a saline solution, although any other fully ionized salt could have been used as the cathode was only used as the return electrode.

### Platform I in Study

Backing: ethylene vinyl acetate (“EVA”) (0.10 mm (4.0 mil)+0.01 mm (0.4 mil)) coated with polyisobutylene (“PIB”) adhesive (6 mg/cm²), (Adhesive Research of Glen Rock, Pa.). The backing was dimensioned to yield a gap of between 0.0940 cm (0.370 inches) and 0.191 cm (0.375 inches)±0.013 cm (0.005 inches) between the gel electrode and the outer edge of the backing at any given point on the edge of the gel. Excluding the tactile feedback notch and the wings, the tab end of the electrode had a width of 1.14 cm (0.450 inches) to 1.27 cm (0.500 inches)±0.013 cm (0.005 inches).

Tab stiffener: 0.18 mm (7 mil) PET/acrylic adhesive (commercially available from Scapa Tapes of Windsor, Conn.).

Anode Gel Reservoir: 1.0 m (40 mil) high adhesion crosslinked polyvinylpyrrolidone (PVP) hydrogel sheet containing: 24% wt.±1% wt. PVP; 1% wt.±0.05% wt. Phenonip; 0.06% wt. NaCl to volume (QS) with purified water (USP).

The anode gel reservoir was circular, having a diameter of 2.52 cm (0.994 inches)±0.013 cm (0.005 inches) and has a volume of about 0.8 mL (0.7 g). The reservoir was loaded by placing 334 mg of an anode loading solution, onto the reservoir and allowing the solution to absorb.

Cathode Reservoir: The unloaded cathode gel consisted of a 1.0 mm (40 mil) high adhesion polyvinylpyrrolidone (PVP) hydrogel sheet containing: 24%±1% wt. PVP, 1% Phenonip antimicrobial, 0.06% wt. NaCl and purified water (Hydrogel Design Systems, Inc.). The cathode reservoir was loaded by placing 227 mg of cathode loading solution onto the surface of the unloaded cathode reservoir and allowing it to fully absorb.

Within-lot variation in solution doses and composition typically is +5%, but has not been analyzed statistically.

Release cover: 0.19 mm (7.5 mil)±0.0095 mm (0.375 mil) polyethylene terephthalate glycolate (PETG) film with silicone coating (Furon 7600 UV-curable silicon). Inside of the release cover are absorbent pads that are ultrasonically bonded to the cover as shown in FIG. 7A.

Electrode Assembly: The electrode was assembled substantially as shown in the figures, with the anode and cathode reservoirs laminated to the electrodes. The tab stiffener was attached to the tab end of the backing of electrode assembly on the opposite side of the backing 3.8 cm (1.5 inch) long from the anode and cathode traces. The drugs were added to the unloaded anode reservoir as indicated below.

Packaging: The assembled electrode assembly was hermetically sealed in a foil-lined polyethylene pouch purged with nitrogen gas.

### Example 1

### Degree of Anesthesia and Sensation of Iontophoresis with the Lidocaine/Epinephrine Patch

The present Example addressed the issues of drug concentration and charge density in order to determine the optimal conditions for the iontophoresis of lidocaine HCl and epinephrine. A maximal charge density of 3.4 mA·min/cm² was employed.

The iontophoretic device used in this Example was the device as described in Patch Fabrication Platform III herein above. However, it should be understood that the anode reservoir-electrode has the same composition, reservoir, lamination structure, and surface areas as the anodes disclosed in Patch Fabrication Platforms I and II. One skilled in the art will appreciate that equivalent results, as described in the present Example, may be obtained using Patch Fabrication Platforms I and II and other variations according to the non-limiting embodiments of the iontophoretic device as described herein.

The overall objectives of this Example were to determine a level of electrical current (charge densities) and epinephrine for a lidocaine HCl 10% patch which would result in minimal dermal effects while achieving clinical local analgesia of acceptable duration within 10 minutes or less of iontophoresis and to assess the sensation associated with iontophoresis at various charge densities under all experimental conditions. More specifically, the objectives of this Example were to assess the following contrasting treatments: (1) effects of a patch containing lidocaine HCl 10% and epinephrine 0% and 0.01% administered with charge densities of 2.5 mA·min/cm² and 3.4 mA·min/cm² versus (2) effects of a patch containing lidocaine HCl 10% and epinephrine 0.1% run at 3.4 mA·min/cm² as a positive control, and (3) the effects of a patch containing lidocaine HCl 10% and epinephrine 0% run with no charge as a negative control.

Sixty subjects were to be enrolled to assess six different treatments administered in pairs. Two treatments were to be evaluated per subject, one on each hand. The treatments consisted of iontophoresis of a patch under various conditions, followed by the cannulation of a vein on the dorsal surface of each hand with a 20 gauge catheter. The six treatments studied in this clinical trial are presented in Table A.

TABLE A
Study Treatments

| | | Charge Density |
|---|---|---|
| Lidocaine HCl | Epinephrine | (mA · min/cm²) |
| 10% | 0% | 0 (Placebo) |
| 10% | 0% | 2.5 |
| 10% | 0% | 3.4 |
| 10% | 0.01% | 2.5 |
| 10% | 0.01% | 3.4 |
| 10% | 0.10% | 3.4 (Positive |
| | | Control) |

The iontophoretic drug delivery system comprised a drug filled anode and saline filled cathode with conductive tabs, as described in Patch Fabrication Platform III, which were connected to a direct current source (controller). The iontophoretic controller provided a constant current and variable voltage source of direct current along with a data acquisition system for capturing current and voltage measurements during the iontophoretic procedure. It consisted of a personal computer (PC) and printer, running MS-DOS and ViewDac software which controlled a Keithley K 575 Data Acquisition System that measured the delivered current and the voltage across the patch, which was recorded in the PC with the controls and two multi-electrode isolated current sources.

The degree of clinical analgesia was determined by cannulation pain using a Visual Analogue Scale (VAS), as described above. Pain assessment scores ranged from 0 to 10 with 0 representing “no pain” and 10 representing “very severe pain.” The duration and degree of analgesia also were determined by the subject's sensitivity to aesthesiometer filaments as a surrogate measure. The sensitivity to aesthesiometer filaments was measured at Baseline (i.e., prior to iontophoresis), immediately following iontophoresis, and 20 minutes post-iontophoresis (continuous). At Baseline, aesthesiometer monofilaments covering a range of calibration needed to determine 0 to 100% detection (in five applications) were used to test for neuropathy, and persons unable to discriminate the 4.31 monofilament were excluded from the study. Testing was done using five applications of monofilaments to the dorsal surface of both hands, following the manufacturer's instructions (until the filament bent). The number of detections for five applications was noted for each filament for both the ascending and descending gauges. Immediately after application of the patch, just after treatment (t=0) and 20 minutes after iontophoresis (t=20 minutes), aesthesiometer filaments of 1.65 to 6.65 were applied five times within the treatment site. The lowest value filament that was detected three of five times was determined twice.

The Sensation Intensity Scale (SIS) and the Hedonic scale, both continuous scales, were used to measure the sensation of iontophoresis. The SIS scale is a vertical VAS with endpoints of “no sensation” and “extremely intense sensation.” The Hedonic scale is a VAS measuring “unpleasantness” and “pleasantness” of sensation.

Subjects assessed pain from the cannulation procedure using a 100-mm VAS scale, and the results are summarized in Table B. There were statistically significant treatment effects (p=0.0002) in the cannulation pain score. The placebo had the highest pain score, while the positive control (lidocaine HCl 10% with epinephrine 0.1% and a charge density of 3.4 mA·min/cm²) had the lowest pain score. The other four treatments had pain scores between these two that were statistically significantly lower than the score for the placebo treatment (p<0.012) and statistically higher than the score for the positive control group (p<0.048), except for the treatment group that received lidocaine HCl with epinephrine 0.01% and a charge density of 3.4 mA·min/cm² (p<0.068). The scores in the four treatment groups were not statistically significantly different from each other.

TABLE B
Cannulation Pain (VAS)

| Treatment | | | |
|---|---|---|---|
| Lidocaine | | Charge Density | Mean |
| HCl | Epinephrine | (mA · min/cm²) | (mm) |
| 10% | 0% | 0 (Placebo) | 50.35 |
| 10% | 0% | 2.5 | 37.50 |
| 10% | 0.01% | 2.5 | 24.26 |
| 10% | 0% | 3.4 | 35.20 |
| 10% | 0.01% | 3.4 | 21.45 |
| 10% | 0.10% | 3.4 (Positive | 19.10 |
| | | Control) | |

Subjects also assessed analgesia by evaluating their sensitivity to aesthesiometer filaments at three time points: at Baseline, immediately following iontophoresis, and at 20 minutes post-iontophoresis. The surrogate measures for analgesia were the three von Frey deltas, the changes in sensitivity between time points: DELTAI0—the difference in detectable gauge size (detecting at least three of five touches) between the initial aesthesiometer assessment (Baseline) and Time 0 (immediately after iontophoresis); DELTAI20—the difference in detectable gauge size between the initial measurement (Baseline) and Time 20 (20 minutes post-iontophoresis); and DELTA020—the difference in detectable gauge size between DELTAI0 and DELTAI20. A summary of the von Frey data is presented in Table C.

Statistically significant treatment effects were observed for DELTAI0 and DELTAI20 (p=0.0001). The results were similar for both parameters. Between the initial testing with aesthesiometer filaments and testing immediately after iontophoresis, the three treatments with epinephrine had statistically significantly larger deltas, indicating greater analgesia than the three treatments without epinephrine, based on paired comparisons (p<0.01).

The analysis indicated no statistically significant differences in analgesia among the six treatments between the time immediately after iontophoresis and 20 minutes post-iontophoresis DELTA020.

This portion of the study demonstrated that the iontophoresis patch reduced the perceived pain of cannulation during the insertion of a 20-gauge catheter from that experienced with the placebo treatment that used no current.

TABLE C
Von Frey Measurements

| Treatment | | | | | |
|---|---|---|---|---|---|
| | | Charge Density | Mean | | |
| Lidocaine HCl | Epinephrine | (mA · min/cm² ) | DELTAI0 | DELTAI20 | DELTA020 |
| 10% | 0% | 0 (Placebo) | 0.24 | 0.39 | 0.15 |
| 10% | 0% | 2.5 | 0.66 | 0.64 | −0.02 |
| 10% | 0.01% | 2.5 | 0.98 | 0.98 | 0.01 |
| 10% | 0% | 3.4 | 0.73 | 0.34 | −0.36 |
| 10% | 0.01% | 3.4 | 1.25 | 1.33 | 0.08 |
| 10% | 0.10% | 3.4 (Positive | 1.15 | 1.06 | −0.09 |
| | | Control) | | | |

Subjects assessed the sensation associated with iontophoresis using the SIS and the Hedonic scale, as described herein above. The results of the SIS and Hedonic scale are summarized in Table D. Statistically significant treatment effects were seen for the SIS score (p=0.0006). The placebo (no current) had a statistically significantly lower score than all other treatments. Among the treatments where a current was applied, there was no statistically significant difference in the SIS score.

For the Hedonic scores, negative integers (−1 to −10) indicated the severity of unpleasant feelings (lower=worse), positive integers (1 to 10) indicated increasingly pleasant feelings (higher=better), and 0 was a neutral score. The mean score for the placebo treatment was higher (mean score=1.5) than for the other treatment groups (mean scores less than or equal to 0). However, there were no statistically significant differences among the six treatments in the Hedonic scores.

TABLE D
Sensation of Iontophoresis

| Treatment | | | | |
|---|---|---|---|---|
| Lidocaine | | Charge Density | Mean | |
| HCl | Epinephrine | (mA · min/cm²) | SIS Score | Hedonic Score |
| 10% | 0% | 0 (Placebo) | 1.65 | 1.50 |
| 10% | 0% | 2.5 | 7.40 | −0.95 |
| 10% | 0.01% | 2.5 | 6.84 | 0 |
| 10% | 0% | 3.4 | 6.15 | −1.05 |
| 10% | 0.01% | 3.4 | 7.4 | −0.80 |
| 10% | 0.10% | 3.4 (Positive | 6.75 | −0.20 |
| | | Control) | | |

In summary, the iontophoresis patch reduced the perceived cannulation pain from that experienced with the placebo treatment which used no current. The positive control group (lidocaine HCl 10% with epinephrine 0.1% delivered at 3.4 mA·min/cm²) provided the best analgesia during insertion of a 20-gauge catheter. Having epinephrine in the patch increased analgesia as measured by the filament aesthesiometer. The sensation scores increased when a current was applied to the patch. However, there were no differences in the SIS score when a 2.5 mA·min/cm² current was used as opposed to a 3.4 mA·min/cm² current. The Hedonic scores were generally close to 0, indicating that, on average, the sensation was very mild and neither pleasant nor unpleasant.

In addition to the above, there were no statistically significant differences in vasoconstriction among treatments, and the success rate for cannulation was 100% in this study. The ease of threading was rated as equivalent for all treatments including placebo and positive control.

The dermal effects, i.e., erythema and edema, of the treatments were also examined in the study. Dermal effects were assessed both on the anode and cathode sides up to about 24 hours post treatment. Draize scores for both erythema (redness) and edema (swelling) were assessed using a scale from 0 to 4, as described herein above, where 0 represents no erythema or no edema, respectively, and 4 represents severe erythema (beet redness to slight eschar formation) or severe edema (swelling raised more than 1 mm and beyond exposed area), respectively.

Erythema and edema Draize scores for the anode and cathode application sites were recorded at three different times: Time 0 (immediately post-iontophoresis), Time 1 (1 hour post-iontophoresis) and Time 24 (about 24 hours post-iontophoresis). The erythema Draize scores are summarized in Table E. The edema Draize scores are summarized in Table F.

For erythema, at the anode site, statistically significant differences among the treatments in the average scores existed from Time 0 and Time 1 (p=0.0001). For both Time 0 and Time 1, the two highest average erythema scores were associated with treatments in which a current was applied and there was no epinephrine in the patch, i.e., no vasoconstrictor to cause blanching. At the cathode site, there were statistically significant differences among treatments in the averaged scores for Time 0 and Time 1. For the 5 treatments where a current was applied to the patch, the average scores were not statistically significantly different. At Time 24, the average score at both the anode and cathode sites was 0 for all treatments. The average Draize score for erythema was less than 2 at either the anode site or cathode site at Time 0, Time 1, and Time 24.

TABLE E
Draize Erythema Scores

| Treatment | | | Mean | | | | | |
|---|---|---|---|---|---|---|---|---|
| | | Charge Density | Time 0 | | Time 1 | | Time 24 | |
| Lidocaine HCl | Epinephrine | (mA · min/cm²) | Anode | Cathode | Anode | Cathode | Anode | Cathode |
| 10% | 0% | 0 (Placebo) | 0.10 | 0.15 | 0.25 | 0.15 | 0.00 | 0.00 |
| 10% | 0% | 2.5 | 0.75 | 1.40 | 1.45 | 1.10 | 0.00 | 0.00 |
| 10% | 0.01% | 2.5 | 0.05 | 1.21 | 0.16 | 1.05 | 0.00 | 0.00 |
| 10% | 0% | 3.4 | 0.90 | 1.40 | 1.90 | 1.05 | 0.00 | 0.00 |
| 10% | 0.01% | 3.4 | 0.00 | 1.15 | 0.45 | 0.90 | 0.00 | 0.00 |
| 10% | 0.10% | 3.4 (Positive Control) | 0.05 | 1.60 | 0.50 | 1.30 | 0.00 | 0.00 |

TABLE F
Draize Edema Scores

| Treatment | | | Mean | | | | | |
|---|---|---|---|---|---|---|---|---|
| | | Charge Density | Time 0 | | Time 1 | | Time 24 | |
| Lidocaine HCl | Epinephrine | (mA · min/cm²) | Anode | Cathode | Anode | Cathode | Anode | Cathode |
| 10% | 0% | 0 (Placebo) | 0.00 | 0.00 | 0.15 | 0.00 | 0.15 | 0.00 |
| 10% | 0% | 2.5 | 0.00 | 0.20 | 0.65 | 0.05 | 0.15 | 0.00 |
| 10% | 0.01% | 2.5 | 0.00 | 0.16 | 0.16 | 0.05 | 0.11 | 0.00 |
| 10% | 0% | 3.4 | 0.05 | 0.15 | 0.55 | 0.05 | 0.10 | 0.00 |
| 10% | 0.01% | 3.4 | 0.00 | 0.10 | 0.10 | 0.10 | 0.00 | 0.00 |
| 10% | 0.10% | 3.4 (Positive Control) | 0.00 | 0.15 | 0.25 | 0.05 | 0.00 | 0.00 |

For edema, at the anode site, there were no statistically significant differences in the average edema scores among the six treatments for Time 0 and Time 24. Statistical differences at Time 1 did exist (p=0.0427). The treatment with lidocaine HCl (10%) and no epinephrine delivered at a charge density of 2.5 mA·min/cm² had the highest average score, while the treatment with lidocaine HCl (10%) and epinephrine (0.01%) delivered at a charge density of 3.4 mA·min/cm² had the lowest score. The two highest scores were for treatments in which current was applied and there was no epinephrine in the patch. At the cathode site, there were no statistically significant differences among treatments at any of the three time points. At Time 24, the average score at both the anode and cathode sites was about 0 for all treatments. The average Draize score was less than 1 for edema at either the anode site or cathode site at Time 0, Time 1, and Time 24.

### Example 2

### Degree of Anesthesia and Sensation of Iontophoresis with the Lidocaine/Phenylephrine Patch

In addition to the above, a small study (n=3) was conducted, testing iontophoretic systems prepared as described above in this Example, but containing 100 mg lidocaine HCl with 0 mg, 0.1 mg, 1 mg, 5 mg or 10 mg phenylephrine. Results indicated that the devices containing both lidocaine HCl and phenylephrine produced generally superior anesthesia as compared to control devices, as determined by a filament aethesiometer as described herein, and iontophoretic sensation was generally acceptable for those devices.

Whereas particular embodiments of the invention have been described herein for the purpose of illustrating the invention and not for the purpose of limiting the same, it will be appreciated by those of ordinary skill in the art that numerous variations of the details, materials and arrangement of parts may be made within the principle and scope of the invention without departing from the invention as described in the appended claims.

We claim:

1. An iontophoresis electrode assembly comprising an anode assembly comprising a pre-loaded hydrogel drug reservoir in electrical communication with a first electrode, the reservoir comprising:
(a) a vasoconstrictor; and
(b) an anesthetic,
the iontophoresis electrode assembly producing clinically acceptable dermal anesthesia and sensation at a treated site as measured by at least about a 50% reduction of dermal sensitivity to an applied force and producing at least one of:
(a) a von Frey score of at least a DELTAI0 of 1.15;
(b) a hedonic score of greater than about −1.5 on a VAS ranging from −10 to 10;
(c) a pain cannulation score of less than about 51 mm on a VAS ranging from 0 to 100 mm;
(d) a Sensation Intensity Scale score of less than about 7.4 on a VAS ranging from 0 to 10;
(e) a Draize score for erythema of less than about 2 on a scale from 0 to 4, and resolving in less than about 48 hours; and
(f) a Draize score for edema of less than about 1 on a scale from 0 to 4, and resolving in less than about 48 hours,
with an applied charge density of between about 1.5 mA·min/cm² and about 4.2 mA·min/cm² that is applied for less than about 10 minutes.

2. The electrode assembly of claim 1, wherein the anesthetic is lidocaine and the vasoconstrictor is epinephrine.

3. The electrode assembly of claim 2, wherein the pre-loaded drug reservoir comprises lidocaine in an amount greater than about 2% by weight of the reservoir.

4. The electrode assembly of claim 3, wherein the pre-loaded drug reservoir comprises lidocaine in an amount of about 10% by weight of the reservoir.

5. The electrode assembly of claim 2, wherein the pre-loaded drug reservoir comprises epinephrine in an amount greater than about 0.005% by weight of the reservoir.

6. The electrode assembly of claim 2, wherein the pre-loaded drug reservoir comprises epinephrine in an amount greater than about 0.01% by weight of the reservoir.

7. The electrode assembly of claim 2, wherein the pre-loaded drug reservoir comprises epinephrine in an amount between about 0.01% by weight of the reservoir and 0.3% by weight of the reservoir.

8. The electrode assembly of claim 2, wherein the pre-loaded drug reservoir comprises epinephrine in an amount of about 0.1% by weight of the reservoir.

9. The electrode assembly of claim 2, wherein the pre-loaded drug reservoir comprises epinephrine in an amount between about 0.01% by weight of the reservoir and about 0.3% by weight of the reservoir and lidocaine in an amount greater than about 2% by weight of the reservoir.

10. The electrode assembly of claim 1, wherein the pre-loaded drug reservoir further comprises an alkaline metal halide salt in an amount between about 0.001% by weight of the reservoir and 1.0% by weight of the reservoir.

11. The electrode assembly of claim 1, wherein the pre-loaded drug reservoir comprises an alkaline metal halide salt in an amount of about 0.06% by weight of the reservoir.

12. The electrode assembly of any of claims 10 and 11, wherein the alkaline metal halide salt is sodium chloride.

13. The electrode assembly of claim 1, the electrode assembly producing a sensation score on a hedonic scale of greater than about −1.5 with an applied charge density between about 1.5 mA·min/cm² and about 4.2 mA·min/cm².

14. The electrode assembly of claim 2, wherein the epinephrine in the drug reservoir degrades to no less than 85% of original levels for at least 24 months at 25° C.

15. The electrode assembly of claim 1, further comprising a cathode assembly comprising a second electrode and a return hydrogel in electrical communication with the second electrode, wherein the first electrode and second electrode are attached to a backing.

16. The electrode assembly of claim 15, further comprising an electrically conductive anode trace attached to the backing and electrically connected to the first electrode; and an electrically conductive cathode trace attached to the backing and electrically connected to the second electrode.

17. The electrode assembly of claim 16, wherein the first electrode and the second electrode and the anode and cathode traces comprise silver/silver chloride-containing ink.

18. The electrode assembly of claim 15, wherein the first electrode and the second electrode are silver/silver chloride electrodes.

19. The electrode assembly of claim 1, wherein the drug reservoir comprises a hydrogel.

20. The electrode assembly of claim 19, wherein the hydrogel is polyvinyl pyrrolidone.

21. The electrode assembly of claim 20, wherein the hydrogel comprises about 15% to about 17% by weight polyvinyl pyrrolidone.

22. The electrode assembly of claim 2, wherein the drug reservoir comprises lidocaine in an amount from about 2% by weight to about 12% by weight of the reservoir and epinephrine in an amount from about 0.001% by weight to about 0.3% by weight of the reservoir.

23. The electrode assembly of claim 22, wherein the reservoir has a volume of about 0.85 mL and comprises about 100 milligrams lidocaine HCl and about 1.05 milligrams epinephrine as free base.

24. The electrode assembly of claim 22, wherein the hydrogel of the drug reservoir has a thickness ranging from 0.089 cm to 0.114 cm thick.

25. The electrode assembly of claim 2, wherein the reservoir comprises lidocaine HCl and epinephrine bitartrate in a mass ratio of about 50:1 to about 1000:1.

26. The electrode assembly of claim 25, wherein the reservoir comprises lidocaine HCl and epinephrine bitartrate in a mass ratio of about 70:1 to about 125:1.

27. The electrode assembly of claim 2, wherein the reservoir further comprises at least one of parabens, sodium metabisulfite, a chelating agent, citric acid, glycerin and sodium chloride.

28. The electrode assembly of claim 2, wherein the anode assembly is prepared by a process comprising contacting an unloaded reservoir containing from about 0.001% by weight to about 1.0% by weight sodium chloride with a solution containing the lidocaine and the epinephrine.

29. A method of producing local anesthesia in a patient, comprising:
applying a charge density of at least about 1.5 mA·min/cm² for at least about 5 minutes to an electrically assisted drug delivery system comprising an anode assembly, the anode assembly including a pre-loaded hydrogel drug reservoir in electrical contact with the patient, the drug reservoir comprising an anesthetic and a vasoconstrictor, the electrically assisted drug delivery system producing clinically acceptable dermal anesthesia and sensation at a treated site as measured by at least about a 50% reduction of dermal sensitivity to an applied force and producing at least one of:
(a) a von Frey score of at least a DELTAI0 of 1.15;
(b) a hedonic score of greater than about −1.5 on a VAS ranging from −10 to 10;
(c) a pain cannulation score of less than about 51 mm on a VAS ranging from 0 to 100 mm;
(d) a Sensation Intensity Scale score of less than about 7.4 on a VAS ranging from 0 to 10;
(e) a Draize score for erythema of less than about 2 on a scale from 0 to 4, and resolving in less than about 48 hours; and
(f) a Draize score for edema of less than about 1 on a scale from 0 to 4, and resolving in less than about 48 hours.

30. The method of claim 29, wherein the charge density is between about 1.5 mA·min/cm² and about 4.2 mA·min/cm².

31. The method of claim 29, wherein the charge density is about 3.4 mA·min/cm².

32. The method of claim 29, wherein the charge density is applied for from about 5 to about 20 minutes.

33. The method of claim 32, wherein the charge density is applied for about 20 minutes.

34. The method of claim 29, wherein a non-necrotizing amount of the vasoconstrictor is administered to the patient.

35. The method of claim 29, wherein the ease of catheterization is the same as without treatment and catheterization is less painful that without treatment.
